(19) United States
(12) Patent Application Publication
(10) Pub. No.: US 2006/0292554 A1
(43) Pub. Date: Dec. 28, 2006

(54) Major coat protein variants for C-terminal and bi-terminal display

(75) Inventors: Heike A. Held, Oakland, CA (US); Sachdev S. Sidhu, San Francisco, CA (US)

Correspondence Address: MERCHANT & GOULD PC, P.O. BOX 2903, MINNEAPOLIS, MN, 55402-0903, US

(73) Assignee: GENENTECH, INC., SOUTH SAN FRANCISCO, CA (US)

(21) Appl. No.: 11/131,109

(22) Filed: May 17, 2005

Related U.S. Application Data
(60) Provisional application No. 60/571,992, filed on May 18, 2004.

(51) Int. Cl. C40B 40/02; C40B 50/06; C12Q 1/70; C07K 14/005

(52) U.S. Cl. 435/5; 435/69.3; 435/456; 435/235.1; 435/325; 530/350

(57) ABSTRACT

The invention provides compositions and methods for enhanced display of heterologous proteins on viral particles.

### RELATED APPLICATIONS

This application is a non-provisional application filed under 37 CFR 1.53(b) (1), claiming priority under 35 USC 119(e) to provisional application No. 60/571,992 filed May 18, 2004, the contents of which are incorporated herein in their entirety by reference.

### BACKGROUND

The filamentous M13 bacteriophage consists of a single-stranded DNA core surrounded by a proteinaceous coat (1). The ends of the virus are capped by minor coat proteins, but the vast majority of the coat consists of several thousand copies of the gene-8 major coat protein (protein-8, P8). P8 molecules cover the length of the filament in a repeating array with N-termini exposed on the surface and C-termini buried in the core (FIG. 1). Assembly of filamentous phage is interesting as both a model system for the study of macromolecular assembly (2; 3; 4), and for its practical implications in phage display technology (5).

Phage display is a powerful approach to protein engineering (6; 7; U.S. Pat. No. 5,780,279; WO00/06717). The method stems from the observation that gene fragments fused to M13 coat protein genes produce fusion proteins that become incorporated into phage coats encapsulating the encoding DNA (6). Displayed polypeptides can be selected from large pools of variants, and polypeptide sequences can be readily deduced from the DNA. In this way, peptides or proteins with new or improved functions can be rapidly “evolved” through in vitro selections from phage-displayed libraries.

Phage display was first demonstrated with M13 bacteriophage (6) and the filamentous phage remains a workhorse for the technology (6; 7). Particularly robust phage display systems have been developed using hybrid phage particles that contain all of the wild-type (wt) coat proteins and an additional fusion protein for display. This has been achieved by the use of phagemids which contain coat protein fusion genes for polypeptide display, but can only be packaged into phage particles in the presence of helper phage that provide all of the proteins necessary for viral assembly (8; 9). The resulting hybrid phage contain both wt coat proteins from the helper phage and fusion coat proteins from the phagemid. The heterologous polypeptide is displayed on the phage surface, but the deleterious effects of the fusion are attenuated by the presence of wt coat proteins from the helper phage. Phagemid systems have been particularly useful for extending the utility of P8-based display systems (5; 10; 11; 12). In the absence of additional wt P8, phage become unstable when peptides greater than ten residues are fused to P8 (13). However, by providing an abundant supply of wt P8 in trans from a helper phage, hybrid phage can be made to display even large proteins, although the levels of display decrease with increasing fusion size and most large proteins are displayed at monovalent levels (10; 14).

Because the phage coat contains several thousand copies of P8, each protein makes only a miniscule contribution to the structural integrity of the phage particle. Thus, in a phagemid system where the fusion-P8 moiety is present as a minor component of the coat, the recombinant P8 has proven remarkably tolerant to mutations. In fact, alanine-scanning mutagenesis studies have shown that only a small subset of the P8 side chains are required for efficient incorporation into the wt coat (15). The extreme sequence malleability of P8 has been exploited to develop improved phage display scaffolds, since certain mutations are not only tolerated but also improve heterologous protein display (10). It has also been shown that, although the P8 C-terminus is buried in the particle core, C-terminal fusions can be displayed (16), albeit at levels significantly lower than those achieved with fusions to the solvent exposed N-terminus.

Despite significant advances in the phage display technology, there remains a significant need to enhance phage display generally, and more specifically C-terminal display. Furthermore, there has been no report of success in achieving optimal bi-terminal display.

### SUMMARY OF THE INVENTION

Conventional phage display methods use wild type coat protein sequences, presumably to enhance stability of the phage particles and to ensure proper incorporation of fusion proteins into the coat of phage particles. Attempts have been made to find major coat protein variants capable of enhanced display or displaying in unconventional orientations (such as C-terminal display). In an attempt to optimize C-terminal and/or bi-terminal display, major coat protein variants were generated and are described herein. Herein, over 600 gp8 coat protein variants are exemplified based on selection for display of polypeptides fused to their C-termini. These variants also provide for generation of improved scaffolds for C-terminal phage display and bi-terminal phage display (i.e., simultaneous display on both C and N termini of a viral coat protein). The invention provides compositions, methods, and kits and articles of manufacture comprising and/or related to the use of such.

In one aspect, the invention provides a fusion protein comprising a heterologous polypeptide fused to a major coat protein of a virus, wherein the major coat protein is a variant of a wild type major coat protein of the virus and is capable of C-terminal display of the heterologous polypeptide at a display level more than about 30, 40, 50, 75 or 100 times that of a corresponding coat protein comprising a wild type sequence.

In another aspect, the invention provides a fusion protein comprising a major coat protein of a virus fused on its N-terminus to a first heterologous polypeptide and on its C-terminus to a second heterologous polypeptide, wherein the major coat protein is a variant of a wild type major coat protein of the virus. In one embodiment, the variant coat protein is capable of C-terminal display of the heterologous polypeptide at a display level more than about 30, 40, 50, 75 or 100 times that of a corresponding coat protein comprising a wild type sequence. In one embodiment, the first and second heterologous polypeptides comprise different sequences. In one embodiment, the first and second heterologous polypeptides comprise/exhibit complementary biochemical functions, for example, enzyme-substrate/product functions, receptor-ligand functions, etc.

In one embodiment, a fusion protein is in a virus particle. In one embodiment, a fusion protein is incorporated in a virus particle coat. In some embodiments of fusion proteins comprising a first and second heterologous polypeptide, the fusion protein displays the first and second polypeptides on the surface of a virus particle.

Various coat protein variants are described herein (e.g., in the form of fusion proteins as described herein) that exhibit advantages with respect to phage display of heterologous polypeptides, in particular C-terminal and bi-terminal display. In various embodiments, a variant coat protein comprises a sequence selected from those listed in FIG. 2, 4, 5 and/or 6B. In one embodiment, a variant coat protein comprises at least one, two, three, four or five substitution in any of positions 1-50, wherein the substitution(s) is with a mutated residue in any of the sequences listed in FIG. 2, 4, 5 and/or 6B. In one embodiment, the variant protein comprises a substitution at one or more of positions 1-50 of the corresponding wild type coat protein, wherein the substitution is with the amino acid indicated as having at least 2, 4, 5, 7, 10 percent occurrence in Table 2. In one embodiment, position 40 of a variant coat protein comprises a positively charged residue (e.g., lysine). In one embodiment, position 43 and/or 44 of a variant coat protein comprises a positively charged residue (e.g., lysine). In one embodiment, positions 43 and/or 44 of a variant coat protein comprise a hydrophobic residue. In one embodiment, position 40 of a variant coat protein comprises a positively charged residue and positions 43 and/or 44 of the variant coat protein comprise a hydrophobic residue. In one embodiment, position 40, 43 and/or 44 of a variant coat protein comprises a positively charged residue (e.g., lysine). In one embodiment of these variant coat proteins, position 48 is not lysine. In one embodiment, the variant coat protein comprises a hydrophobic epitope comprising one or more (e.g., one, two, three or all) of positions 39, 41, 42 and 45. In one embodiment, the variant coat protein further comprises a hydrophobic epitope comprising one or more (e.g., one, two, three or all) of positions 25, 26, 28 and 29. In one embodiment, a variant coat protein has about 2-50, 5-45, 10-30, 15-25 altered residues relative to the corresponding wild type coat protein sequence. In one embodiment, the variant coat protein is fused to a heterologous polypeptide which is an antibody or a fragment thereof, a cytokine, a cytokine receptor, an enzyme/substrate, an inhibitor/target polypeptide, a receptor/ligand, etc. In one embodiment wherein there is a first and second heterologous polypeptide, the polypeptides are labeled with a suitable moiety that results in a detectable signal when the first and second heterologous polypeptides interact in accordance with a selection/screening criterion (e.g., when the two heterologous polypeptides are in a suitable structure-function relationship and/or proximity to each other).

In one aspect, one or more (e.g., two, three, four, up to all) of positions 46-50 of a variant coat protein are deleted or substituted. In one embodiment of variant coat proteins wherein one or more (up to all) of positions 46-50 are substituted, the substitution is with an amino acid compatible with formation of a flexible linker sequence (e.g., glycine).

In one embodiment, a fusion protein of the invention further comprises a linker sequence between the C-terminus of a variant coat protein and the N-terminus of a heterologous polypeptide. Accordingly, in some embodiments, a C-terminally fused heterologous polypeptide is indirectly linked to the C-terminus of a variant coat protein through a linker sequence comprising at least about 5, 7, 9, 11, 13, 15, 17, 20 amino acid residues. In one embodiment, the linker sequence has 10 amino acids and comprises residues 51-60 of the sequence indicated as “wt” in Table 2 (i.e., AWEENIDSAP). In one embodiment, the linker sequence comprises a substitution in at least one position with an amino acid indicated for that position in Table 2, FIG. 2, 4 and/or 5. In one embodiment, the substitution is with an amino acid indicated as having at least 2, 3, 4, 5, 7, 9, 10 percent occurrence in Table 2.

Coat proteins can be found in a number of viruses, including but not limited to filamentous phage, lambda phage, Baculovirus, T4 phage and T7 phage. For example, a coat protein can be that of a filamentous phage, wherein the coat protein is gpVIII.

In one aspect, the invention provides a polynucleotide encoding a polypeptide of the invention. In one embodiment, the polynucleotide is replicable expression vector comprising a nucleic acid sequence encoding a polypeptide of the invention, e.g. wherein the vector comprises a gene fusion, wherein the gene fusion encodes a fusion polypeptide of the invention.

In one aspect, the invention provides a library comprising a plurality of the polynucleotides of the invention, e.g. replicable expression vectors of the invention wherein the expression vectors encode a plurality of fusion proteins.

In one aspect, the invention provides a host cell comprising a polynucleotide (e.g., a vector) of the invention.

In one aspect, the invention provides a virus or a plurality of virus particles (e.g., a library) displaying a fusion polypeptide of the invention on the surface thereof.

In one aspect, the invention provides a method comprising: generating a population of virus (phage or phagemid) particles displaying a plurality of the fusion polypeptides of the invention; contacting the virus (phage or phagemid) particles with a target molecule or substance; and separating particles having a desired selection characteristic from those that do not.

In one aspect, the invention provides a method comprising generating a composition comprising a plurality of replicable expression vectors, each expression vector comprising a transcription regulatory element operably linked to a gene fusion encoding a fusion polypeptide, where the gene fusion comprises a first gene encoding a first polypeptide and a second gene encoding a variant viral major coat protein, where the composition comprises a plurality of first genes encoding a plurality of variant first polypeptides (e.g., differeing at one or more positions due to, for example, mutagenesis). In one embodiment, the method further comprises transforming suitable host cells with the plurality of vectors and culturing the transformed cells under conditions suitable to form the fusion polypeptides. In some embodiments, the vector is phage or phagemid DNA and the culturing is sufficient to form phage or phagemid particles which display fusion polypeptides on the surfaces thereof. In one embodiment, the method further comprises contacting the phage or phagemid particles with a target molecule so that at least a portion of the particles exhibit a desired selection characteristic (e.g., binding at a desired affinity, signal generation/reduction (e.g., in bi-terminal display as described herein)), and separating particles having desired selection characteristic from those that do not.

In one aspect, the invention provides a composition comprising a polypeptide and/or polynucleotide of the invention. In one aspect, the invention provides a kit comprising a polypeptide and/or polynucleotide of the invention, which in some embodiments further comprises instructions for using the polypeptide and/or polynucleotide of the invention (e.g., in methods of the invention). In one aspect, the invention provides an article of manufacture comprising a polypeptide and/or polynucleotide of the invention.

### BRIEF DESCRIPTION OF THE DRAWINGS

FIG. 1. The filamentous phage coat is shown as a repeating array of P8 molecules. The P8 molecules are shown as ribbons. The P8 N-termini are exposed on the particle surface while the C-termini are buried at the core. The wt P8 of the Ff phage coat (PDB entry 1IFJ) 21 is shown in CPK representation. M13 P8 differs from Ff P8 in only one position (Asn12 in M13 P8 is an Asp in Ff P8). This figure and FIG. 3 were generated using Insight II (Accelrys, San Diego).

FIGS. 2A & B. Sequence diversity and Shannon entropy of P8-C. For each position in wt P8-C, the most frequent amino acid types observed in the sequence diversity database (Table 2) are shown in rank order of frequency. All amino acid types that occurred with at least 5% frequency are shown. The data were used to calculate Shannon entropy values (see Materials and Methods). Letters in bold indicate wild type amino acid.

FIG. 3. Mapping of Shannon entropy values according to FIG. 2 onto the structure of P8. While the C terminal region of the α-helical P8 is buried completely in the phage coat, only one face of the N-terminal portion is buried. Panel (A) shows the buried face, while in panel (B), rotation by 180° reveals the exposed face.

FIG. 4. First generation P8-C variants selected for enhanced C-terminal display. Sequences are shown for clones isolated following eight rounds of selection for C terminal display. For the selected clones (S1, S2, etc.), only the sequences that differed from wt are shown, and the wt sequence is shown at the top along with the amino acid numbering. The numbers in parentheses indicate the number of times a sequence was observed in cases where the sequence was not unique. Asterisks indicate sequences where multiple clones were not identical but contained a common three residue motif at positions 47-49 (e.g. clone S9 represents eight unique clones that contained a common CGG sequence at positions 47-49 but differed at positions 50-53). For some clones, the display enhancement (DE) relative to wt P8-C was determined.

FIG. 5. Combination of first generation P8-C mutations. Mutations in different regions of P8-C were combined with the mutations in P8-C-S14. For each combined mutant (M1, M2, etc.), only the sequences that differed from wt are shown, and the wt sequence is shown at the top along with the amino acid numbering. The source of each set of mutations (FIG. 4) is given in parentheses. The display enhancement (DE) relative to wt P8-C is shown for each variant.

FIGS. 6A & B. Second generation P8-C variants. Variants were selected from a second generation library in which the N-terminal half of P8 was randomized in the background of first generation variant P8-C-S14. (A) Design of the second generation library. The wt P8 sequence from positions 6-24 was randomized with tailored degenerate codons (italics). At each position, a tailored codon was designed to encode mainly amino acids frequently observed amongst P8-C variants selected for C-terminal display (shown below the codon in bold text), but additional amino acids (plain text) were included at some positions due to the redundancy of the genetic code. (B) Second generation P8-C variants exhibit enhanced C-terminal display. The display enhancement (DE) relative to wt P8-C is shown to the right of each sequence selected from the second generation library. Dashes indicate sequences that were conserved as the wt. DNA degeneracies are represented in the IUB code (B=T/C/G, D=T/A/G, H=T/C/A, K=T/G, M=C/A, N=T/C/A/G, R=A/G, S=G/C, V=C/A/G, W=T/A, Y=T/C).

FIG. 7. Simultaneous display of N- and C-terminal fusions. (A) Phage ELISAs were used to detect the display of a gDtag epitope (filled symbols) or an Erbin PDZ ligand (open symbols) fused to the N- or C-terminus, respectively. The peptides were fused simultaneously to either wt P8-C (circles) or the variant P8-C-S16 (squares). (B) Western blot analysis of phage incorporating P8-C variants. Purified phage (˜10¹⁰ colony forming units) were subjected to SDS-PAGE and a blot was probed with an anti-gDtag antibody. Phage were produced from E. coli cultures coinfected with M13K07 helper phage and phagemids encoding for various P8 moieties with gDtag epitopes fused to their N-termini for detection. The following P8 moieties were analyzed: lane 1, P8-C; lane 2, P8-C-S14; lane 3, P8-C-S16; lane 4, P8-C-S27, lane 5, wt P8 (no C-terminal fusion).

### DETAILED DESCRIPTION OF THE INVENTION

### Definitions

Display level of a fusion protein is “x times” that of a reference amount refers to the relative amount of detectable viral surface display of a heterologous polypeptide effected by fusion protein comprising a variant coat protein of the invention compared to fusion protein comprising the corresponding wild type protein. Amount of display can be qualitative or quantitative. Relative amounts of display can also be expressed in terms of “fold” difference or “display enhancement” (as described in Examples). Examples of measurement of display level include that which is based on number of copies of the heterologous polypeptide present on a viral surface that is capable of binding to a corresponding target molecule. Another measurement can be based on quantitation by electron microscopy. Yet another measurement is based on phage ELISA as described in the Examples.

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, as well as antibody fragments (e.g., Fab, F(ab′)₂, scFv and Fv), so long as they exhibit the desired biological activity (e.g., antigen binding). An affinity matured antibody will typically have its binding affinity increased above that of the isolated or natural antibody or fragment thereof by from 2 to 500 fold. Preferred affinity matured antibodies will have nanomolar or even picomolar affinities to the receptor antigen. Affinity matured antibodies are produced by procedures known in the art. Marks, J. D. et al. Bio/Technology 10:779-783 (1992) describes affinity maturation by VH and VL domain shuffling. Random mutagenesis of CDR and/or framework residues is described by: Barbas, C. F. et al. Proc Nat. Acad. Sci, USA 91:3809-3813 (1994), Schier, R. et al. Gene 169:147-155 (1995), Yelton, D. E. et al. J. Immunol. 155:1994-2004 (1995), Jackson, J. R. et al., J. Immunol. 154(7):3310-9 (1995), and Hawkins, R. E. et al, J. Mol. Biol. 226:889-896 (1992). Humanized antibodies are known. Jones et al., Nature, 321:522-525 (1986); Reichmann et al., Nature, 332:323-329 (1988); and Presta, Curr. Op. Struct. Biol., 2:593-596 (1992)).

An “Fv” fragment is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” refers to the antibodies described in Zapata et al. Protein Eng. 8(10): 1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

“Cell,” “cell line,” and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is a coat protein which is present in the viral coat at 10 copies of the protein or more. A major coat protein may be present in tens, hundreds or even thousands of copies per virion. As used herein, “coat protein” includes full length (i.e., wild type/native length) and fragments (portions) thereof (so long as the fragment is capable of effecting display of a fusion protein at a desired level as indicated herein).

A “fusion protein” is a polypeptide having multiple (two or more) portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The portions may be linked directly by a single peptide bond or through a peptide linker containing one or more amino acid residues. Generally, the portions and the linker will be in reading frame with each other.

A “mutation” is a deletion, insertion, or substitution of a nucleotide(s) relative to a reference nucleotide sequence, such as a wild type sequence.

A “silent mutation” is a mutation which does not change the amino acid sequence of the translated polypeptide product of a given DNA sequence.

A “non-silent mutation” is a mutation which changes the amino acide sequence of the translated polypeptide product of a given DNA sequence.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adapters or linkers are used in accord with conventional practice.

“Phage display” is a technique by which variant polypeptides are displayed as fusion proteins to a coat protein on the surface of phage, e.g. filamentous phage, particles. A utility of phage display lies in the fact that large libraries of randomized protein variants can be rapidly and efficiently sorted for those sequences that bind to a target molecule with high affinity. Display of peptides and proteins libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 1992, 3:355-362 and references cited therein. In monovalent phage display, a protein or peptide library is fused to a gene III or a portion thereof and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 1991, 3:205-216.

A “phagemid” is a plasmid vector having a bacterial origin of replication, e.g., ColE1, and a copy of an intergenic region of a bacteriophage. The phagemid may be based on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle. Sambrook et al., above, 4.17.

The term “phage vector” means a double stranded replicative form of a bacteriophage containing a heterologous gene and capable of replication. The phage vector has a phage origin of replication allowing phage replication and phage particle formation. The phage is preferably a filamentous bacteriophage, such as an M13, f1, fd, Pf3 phage or a derivative thereof, or a lambdoid phage, such as lambda, 21, phi80, phi81, 82, 424, 434, etc., or a derivative thereof.

“Oligonucleotides” are short-length, single- or double-stranded polydeoxynucleotides that are chemically synthesized by known methods (such as phosphotriester, phosphite, or phosphoramidite chemistry, using solid-phase techniques such as described in EP 266,032 published 4 May 1988, or via deoxynucleoside H-phosphonate intermediates as described by Froehler et al., Nucl. Acids Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides are then purified on polyacrylamide gels.

DNA is “purified” or “isolated” when the DNA is separated from non-nucleic acid impurities. The impurities may be polar, non-polar, ionic, etc.

A chemical group or species having a “specific binding affinity for DNA” means a molecule or portion thereof which forms a non-covalent bond with DNA which is stronger than the bonds formed with other celular components including proteins, salts, and lipids.

A “transcription regulatory element” will contain one or more of the following components: an enhancer element, a promoter, an operator sequence, a repressor gene, and a transcription termination sequence. These components are well known in the art. U.S. Pat. No. 5,667,780.

A “transformant” is a cell which has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

A “variant” or “mutant” of a starting polypeptide, such as a fusion protein or a heterologous polypeptide (heterologous to a phage), is a polypeptide that 1) has an amino acid sequence different from that of the starting polypeptide and 2) was derived from the starting polypeptide through either natural or artificial (manmade) mutagenesis. Such variants include, for example, deletions from, and/or insertions into and/or substitutions of, residues within the amino acid sequence of the polypeptide of interest. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

Generally, a variant coat protein will possess at least 20% or 40% sequence identity and up to 70% or 85% sequence identity, more preferably up to 95% or 99.9% sequence identity, with the wild type coat protein. Percentage sequence identity is determined, for example, by the Fitch et al., Proc. Natl. Acad. Sci. USA 80:1382-1386 (1983), version of the algorithm described by Needleman et al., J. Mol. Biol. 48:443-453 (1970), after aligning the sequences to provide for maximum homology. Amino acid sequence variants of a polypeptide are prepared by introducing appropriate nucleotide changes into DNA encoding the polypeptide, or by peptide synthesis. An “altered residue” is a deletion, insertion or substitution of an amino acid residue relative to a reference amino acid sequence, such as a wild type sequence.

A “functional” mutant or variant is one which exhibits a detectable activity or function which is also detectably exhibited by the wild type protein. For example, a “functional” mutant or variant of a major coat protein is one which is stably incorporated into the phage coat at levels which can be experimentally detected. Preferably, the phage coat incorporation can be detected in a range of about 1 fusion per 1000 virus particles up to about 1000 fusions per virus particle.

A “hyper-functional” mutant or variant is a functional mutant or variant whose activity exceeds that of the wild type. For example, a hyper-functional mutant or variant of a major coat protein is one which is stably incorporated into the phage coat at levels greater than those of the wild type protein in a substantially identical context.

A “hypo-functional” mutant or variant is a functional mutant or variant whose activity is less than that of the wild type. For example, a hypo-functional mutant or variant of a major coat protein is one which is stably incorporated into the phage coat at levels less than those of the wild type protein in a substantially identical context.

A “wild type” sequence or the sequence of a “wild type” protein, such as a coat protein, is the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

### MODES FOR CARRYING OUT THE INVENTION

The M13 major coat protein contains 50 residues which can be divided into three regions: The periplasmic domain contains residues 1 to 20, the transmembrane domain contains residues 21 to 39, and the cytoplasmic domain contains residues 40 to 50 (Marvin, D. A. (1998) Current Opinion in Structural Biology 8:150). The other major coat proteins in Table 1 below have a similar domain structure.

Although it has been reported that fusion proteins of heterologous polypeptides to variants of the major coat proteins of bacteriophage are tolerated in phage display systems (see, e.g., WO00/06717), it is apparent that display levels can be further optimized, in particular for C-terminal display and the hitherto unknown bi-terminal display. In one aspect of the present invention, phage display and selection have been used to obtain virus (e.g., bacteriophage) displaying fusion proteins on the surface thereof where the fusion protein is a heterologeous polypeptide fused to the C-terminus of a virus major coat protein variant (or portion thereof) having one or more amino acid substitutions, deletions or additions, wherein the display level is greater than that achieved with the wild type counterpart coat protein. In another aspect, the invention provides fusion polypeptides capable of achieving bi-terminal display on a viral surface. In one embodiment, fusion polypeptides having a heterologous polypeptide linked to a variant of the coat proteins in the Table 1 below are within the scope of this invention.

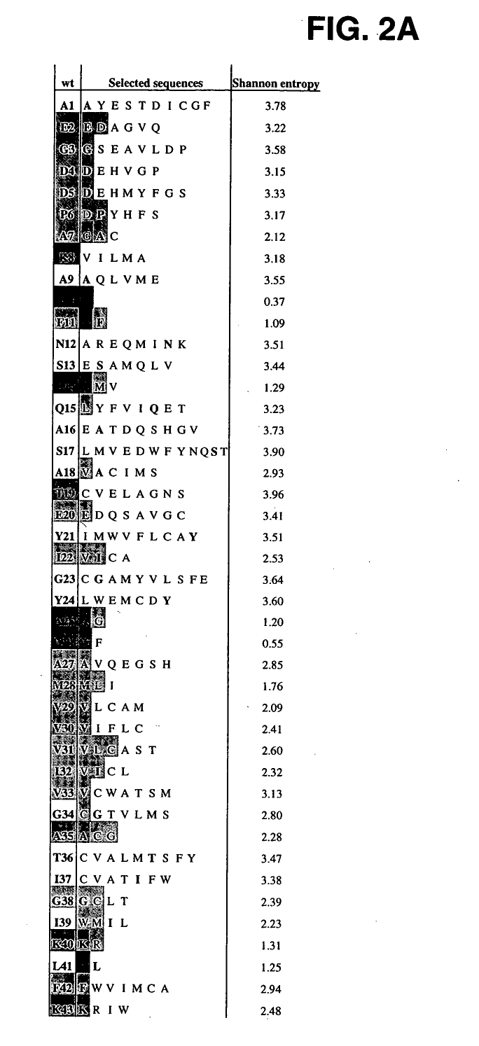

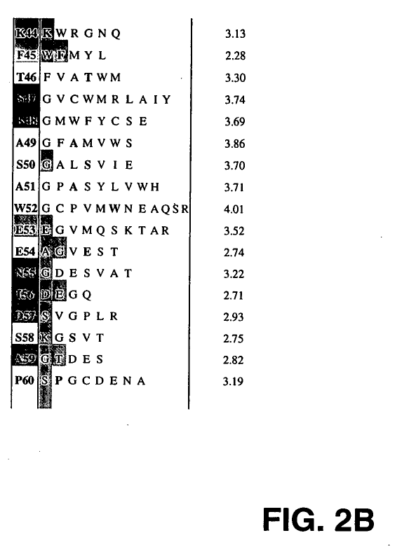

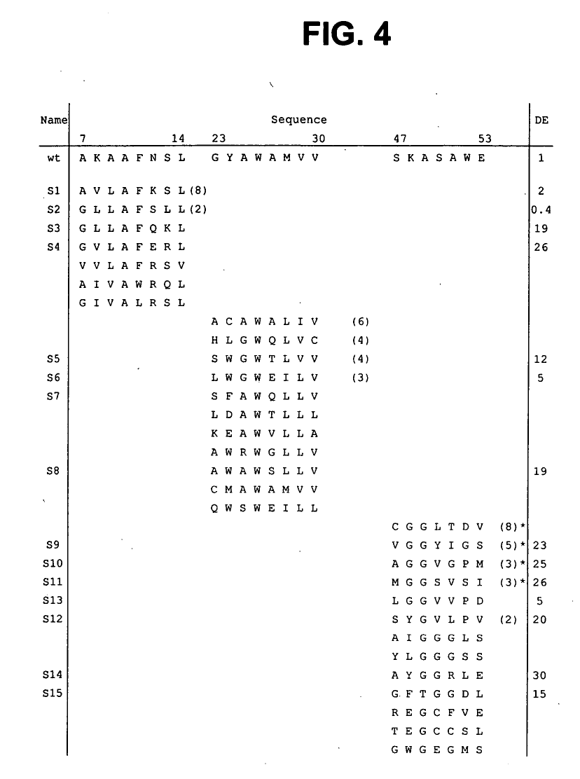

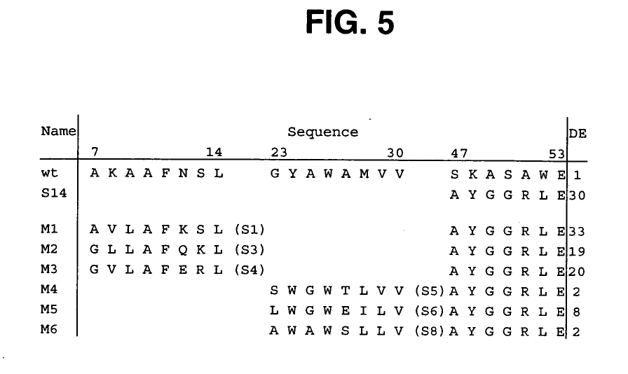

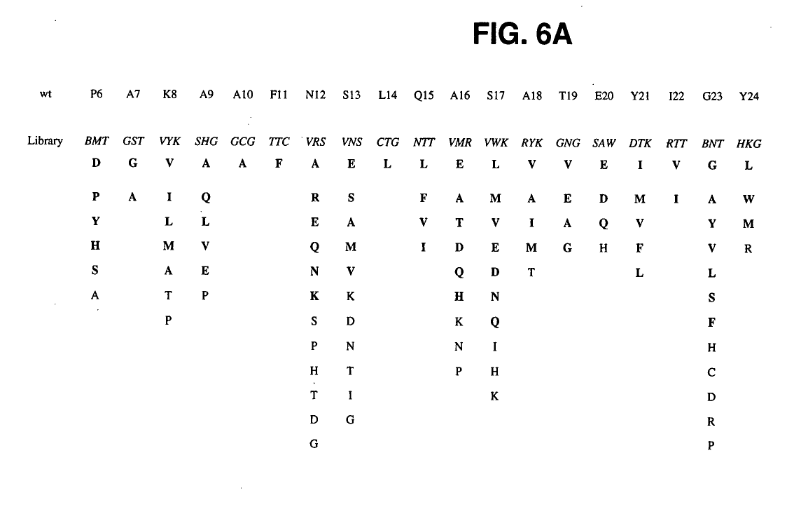

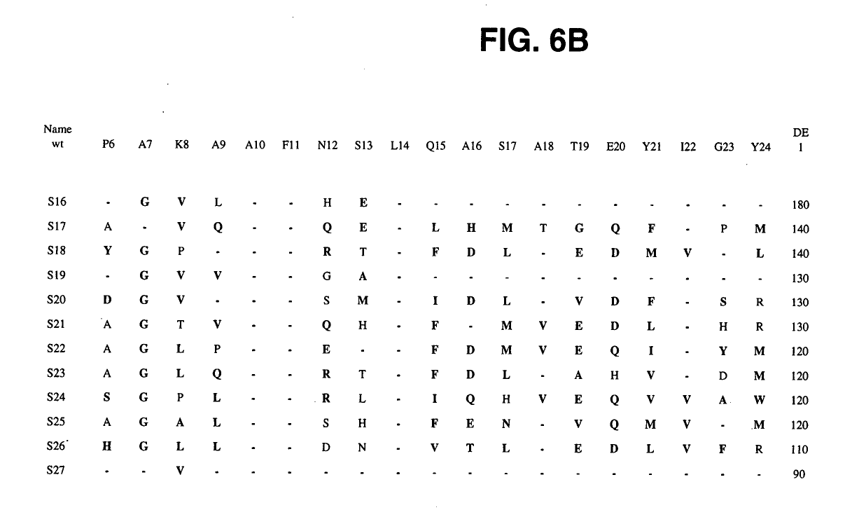

Examples of suitable variants of M13, f1 and fd coat protein VIII include those that contain at least one amino acid residue substitution in Table 2, FIG. 2, FIG. 4, FIG. 5 and/or FIG. 6 indicated as capable of increasing C-terminal and/or bi-terminal display greater than that of the corresponding wild type coat protein (or portion thereof). In the Table and Figures, the letter code refers to amino acid residues as follows: A (Ala) alanine; B (Asx) asparagine or aspartic acid; C (Cys) cysteine; D (Asp) aspartic acid; E (glu) glutamic acid; F (Phe) phenylalanine; G (Gly) glycine; H (His) histidine; I (Ile) isoleucine; K (Lys) lysine; L (Leu) leucine; M (Met) methionine; N (Asn) asparagine; O (Xaa) stop codon; P (Pro) proline; Q (Gln) glutamine; R (Arg) arginine; S (Ser) serine; T (Thr) threonine; V (Val) valine; W (Trp) tryptophan; X (Xaa) unknown or non-standard; Y (Tyr) tyrosine; Z (Glx) gluamine or glutamic acid.

As described herein (see, e.g. Examples), it has been discovered that the amino acid sequence of phage major coat proteins can be modified to produce variants of the major coat protein which are optimized as components of fusion proteins in phage display systems and methods for C-terminal and/or bi-terminal display. In general, fusion polypeptides containing variants of the major coat protein of a bacteriophage influence the ability of phage to display and/or package the fusion polypeptides into complete virus particles (virions) useful in screening/selection methods. That is, variants of the major coat proteins can be used to alter the number of fusion proteins incorporated into a virus particle, and hence display levels. As shown herein, hyper-functional variants of a major coat protein can be used to increase the number of fusion proteins incorporated into a virus particle. Conversely, the data described herein also demonstrate hypo-functional variants that can result in a decrease in fusion protein incorporation. The variant proteins of the invention are particularly useful for tailoring the incorporation of fusion polypeptides into virus particles to achieve a desired level of valency. This is particularly important for fusion polypeptides in which the heterologous polypeptide is relatively large, for example, where the heterologous polypeptide contains 50 or more amino acids, 100 or more amino acids, or 200 or more amino acid residues, and also where the heterologous polypeptide is a protein having secondary and tertiary structure, or the heterologous polypeptide is displayed C-terminally or bi-terminally. The compositions and methods of the invention, therefore, provide a means of overcoming the deficiencies of prior art phage display methods which utilize the major coat protein of a bacteriophage and which often obtain less than optimal incorporation of a fusion polypeptide into the virus coat. The fusion polypeptides of the invention are able to function in known phage display systems by substituting for the conventionally used wild type coat protein fusions with heterologous polypeptides. The fusion polypeptides of the invention will function in a similar manner to conventional fusion proteins in each of the known phage display systems, in which the fusion is with the major coat protein of the virus, further allowing one to select the degree of valency or number of fusion proteins displayed on the surface of the phage with more reliability. For example, the phage and phagemid vectors and the phage display systems described in U.S. Pat. No. 5,223,409; U.S. Pat. No. 5,403,484; U.S. Pat. No. 5,571,689; U.S. Pat. No. 5,750,373, WO00/06717 and U.S. Pat. No. 5,780,279 (and others noted above) can be modified to use the fusion proteins of the invention to improve display of peptides, proteins, antibodies and fragments thereof on the surface of phage. The phage is preferably a DNA phage.

In addition to filamentous phage, the invention is suitable for use in phage display systems using lambda phage, Baculovirus, T4 phage and T7 phage. In each of these display systems, the coat protein used to display a heterologous polypeptide is mutated to form variants of the coat protein using the method of the invention and variants having the desired degree of display (hyper-functional or hypo-functional variants) are selected. The selected variant coat protein is then used to form a fusion protein with a heterologous polypeptide which is to be displayed on the surface of the virus particles. The scope of this invention includes the method(s) of the invention using these phage as well as fusion proteins, replicable expression vectors containing a gene encoding the fusion protein, virus particles containing the fusion proteins or vectors, host cells containing the virus particles, fusion proteins or vectors, libraries containing a plurality of different individuals of these fusion proteins, vectors, virions, cells, etc.

Polypeptides may be displayed on lambdoid phage using coat proteins in either the head or the tail portions of the phage particle (U.S. Pat. No. 5,627,024). Suitable head proteins include proteins pE, pD, pB, pW, pFII, pB* (a cleavage product of pB), pXI, and pX.2; suitable tail proteins include pJ, pV, pG, pM, and pT. The structure and location of these coat proteins is well known. See Georgeopoulos, et al. and Katsura in “Lambda II”, R. W. Hendrix et al. eds. Cold Spring Harbor Laboratory, Cold Spring Harbor, N.Y., 1983. Preferred lambda proteins for use in the invention are the tail coat proteins, particularly pV. U.S. Pat. No. 5,627,024 describes how to display polypeptides on lambda phage, preferably using pV. The fusion proteins of the invention, therefore, include at least a portion of variants of pE, pD, pB, pW, pFII, pB*, pXI, pX.2, pJ, pV, pG, pM, and pT fused to a heterologous polypeptide.

Polypeptides can also be displayed on T4 phage. The structure of the T4 virion is well studied. See Eiserling in “Bacteriophage T4”, C. K. Mathews et al. eds. American Society for Microbiology, Washington, D.C., 1983, pp 11-24. Peptides and full length proteins may be displayed as fusions with the SOC (small outer capsid protein) and the HOC (highly antigenic outer capsid protein) coat proteins of T4 phage. Further, the minor T4 fibrous protein fibritin encoded by the wax (whisker's antigen control) gene can be lengthened at the C terminus with a heterologous polypeptide to form a fusion protein which is displayed on the T4 whisker protein. See Ren, Z-J. et al. (1998) Gene 215:439; Zhu, Z. (1997) CAN 33:534; Jiang, J et al. (1997) can 128:44380; Ren, Z-J. et al. (1997) CAN 127:215644; Ren, Z-J. (1996) Protein Sci. 5:1833; and Efimov, V. P. et al. (1995) Virus Genes 10:173.

T7 phage may also be used to display polypeptides and proteins. Smith, G. P. and Scott, J. K. (1993) Methods in Enzymology, 217, 228-257; U.S. Pat. No. 5,766,905. Commercial kits (T7Select1-1 and T7Select415-1 from Novagen) are available for display of polypeptides as fusion proteins with the 10B capsid protein (397 amino acids) and with the 10A capsid protein (344 amino acids). These systems are easy to use and have the capacity to display peptides up to about 50 amino acids in size in high copy number (415 per phage), and proteins up to about 1200 amino acids in low copy number (0.1-1 per phage). T7 is a double stranded DNA phage that has been extensively studied (Dunn, J. J. and Studier, F. W. (1983) J. Mol. Biol. 166:477-535; Steven, A. C. and Trus, B. L. (1986) Electron Microscopy of Proteins 5:1-35). Phage assembly takes place inside the host (E. coli) cell and mature phage are released by cell lysis. Fusion proteins of heterologous polypeptides to variants of T7 coat proteins, such as 10B and 10A, vectors containing a gene encoding the fusion protein, etc. are within the of the invention. Preferably, fusion proteins are prepared by altering, preferably by mutating to a non-wild type amino acid, one or more of residues 1-348 of capsid protein 10B.

The invention also includes fusion proteins of heterologous polypeptides with Baculovirus coat protein variants. Baculovirus expression vectors, particularly those based on Autographa californica nuclear polyhedrosis virus, are easily generated and are now widely used for the expression of heterologous polypeptides in cultured insect cells and insect larvae (Weyer, U. and Possee, R. D. (1991) J. Gen. Virol. 72:2967). These viruses contain a double stranded, circular genome, where foreign genes can be inserted easily. Tarui, H. et al. (1995) J. Fac. Agr. Kyushu Univ., 40;45. It is possible to display a glycosylated eukaryotic protein on the surface of baculovirus particles, using a fusion with the baculovirus major coat protein gp64 or at least by fusing the heterologous polypeptides to the membrane anchorage domain of gp64 only. The efficiency of various promoters (polyhedrin, basic, gp64-promoter) have been examined, including the “very late” polyhedrin promoter and the “early and late” gp64 promoter. In order to express a foreign gene on the surface of baculoviruses efficiently, it is necessary to choose a regulating promoter, that on one hand will transcribe sufficient amounts of the target protein, and on the other hand start transcription early enough in the viral replication cycle, to guarantee efficient packaging, complete glycosylation and correct folding.

Variants containing about 2-49, about 5-40, or about 7-20, altered residues are possible. Major coat protein variants containing about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 variant residues are possible.

Having obtained a variant major coat protein which improves/tailors display of the heterologous polypeptide on the surface of phage particles, it is then possible to use conventional phage display technologies to construct libraries of variants of the originally displayed heterologous polypeptide and select for a desired property, e.g., binding, enzymatic activity, etc. The fusion protein of the invention, containing a selected variant of the major coat protein of the phage which provides the desired display characteristics, can be used to replace a fusion protein in conventional phage display systems where the conventional fusion protein contains the wild type amino acid sequence of the major coat protein or coat protein fragment. Replacement of the conventional fusion protein with the variant fusion protein of the invention improves the display of heterologous polypeptide in the phage display system. That is, the coat protein portion has been optimized for the polypeptide which is displayed as a fusion protein.

Phage display methods for proteins, peptides and mutated variants thereof, including constructing a family of variant replicable vectors containing a transcription regulatory element operably linked to a gene fusion encoding a fusion polypeptide, transforming suitable host cells, culturing the transformed cells to form phage particles which display the fusion polypeptide on the surface of the phage particle, contacting the recombinant phage particles with a target molecule so that at least a portion of the particle bind to the target, separating the particles which bind from those that do not bind, are known and may be used with the method of the invention. See U.S. Pat. No. 5,750,373; WO00/06717; WO 97/09446; U.S. Pat. No. 5,514,548; U.S. Pat. No. 5,498,538; U.S. Pat. No. 5,516,637; U.S. Pat. No. 5,432,018; WO 96/22393; U.S. Pat. No. 5,658,727; U.S. Pat. No. 5,627,024; WO 97/29185; O'Boyle et al., 1997, Virology, 236:338-347; Soumillion et al., 1994, Appl. Biochem. Biotech., 47:175-190; O'Neil and Hoess, 1995, Curr. Opin. Struct. Biol., 5:443-449; Makowski, 1993, Gene, 128:5-11; Dunn, 1996, Curr. Opin. Struct. Biol., 7:547-553; Choo and Klug, 1995, Curr. Opin. Struct. Biol., 6:431-436; Bradbury and Cattaneo, 1995, TINS, 18:242-249; Cortese et al., 1995, Curr. Opin. Struct. Biol., 6:73-80; Allen et al., 1995, TIBS, 20:509-516; Lindquist and Naderi, 1995, FEMS Micro. Rev., 17:33-39; Clarkson and Wells, 1994, Tibtech, 12:173-184; Barbas, 1993, Curr. Opin. Biol., 4:526-530; McGregor, 1996, Mol. Biotech., 6:155-162; Cortese et al., 1996, Curr. Opin. Biol., 7:616-621; McLafferty et al., 1993, Gene, 128:29-36.

The heterologous polypeptide may be linked to the coat protein or portion thereof through a peptide linker. A linker peptide segment will generally vary in length from about 3 to about 50 amino acid residues, preferably from 5 to 30 residues, more preferably from 10 to 25 residues. In some embodiments, the net charge on the linker segment is positive. The identity of and order of the amino acid residues is optional, although one or more specific sequences of the linker peptide segment will generally provide better display of the heterologous polypeptide. Optimized linker sequences can be obtained by mutating a template linker between the fused protein and the coat protein and selecting linkers which afford the desired level of display. For example, a library of linker segment variants is made by mutating a linker sequence template and the linker sequences which give the best display on phage are selected using phage display selection, for example, an affinity selection for binding to the displayed heterologous polypeptide. Linkers which allow for greater numbers of displayed polypeptides will be selected based on increased affinity for the affinity matrix. See also WO00/06717.

As described herein, one aspect of the invention is the carboxyl (C-terminal) and bi-terminal display of a heterologous polypeptide on the surface of a filamentous phage using protein fusions with a viral coat protein such as protein VIII. C-terminal display has been reported on protein VI of M13 (Jespers, L. et al., 1995, Biotechnology 13:378-382) and on protein VIII (WO00/06717). Jespers et al. state that protein VI is distinct from proteins III and VIII in its ability to allow for the attachment of polypeptides at the C-terminus. The present invention demonstrates that with a suitable coat protein variant, not only is C-terminal display possible, it can be optimized to obtain display levels that exceed that obtained using a wild type coat protein and also in the context of bi-terminal display. The invention, therefore, allows C-terminal and bi-terminal display of heterologous polypeptides or library of polypeptides in a manner useful for use in screening/selection processes.

C-terminal display finds use in a number of settings that would be evident to one skilled in the art. For example, it is useful to display intracellular, such as mammalian intracellular, proteins or fragments thereof and polypeptides which are difficult to display using N-terminal display. C-terminal display can, therefore, be a complementary display technique to N-terminal display. Intracellular proteins may be difficult to display in a correctly folded form using N-terminal display due to the difference in redox environment in which intracellular proteins normally exist relative to the environment in which secreted proteins fold and form disulfide bonds. The cytoplasm is a reducing environment whereas the periplasm is an oxidizing environment. C-terminal heterologous fusion proteins migrate to the periplasm as in normal phage particle assembly. However, since the heterologous polypeptide remains on the intracellular side of the periplasmic membrane, an intracellular polypeptide may correctly fold prior to incorporation into a phage particle. During assembly of the phage or phagemid particle, the C-terminal fusion protein is incorporated into the particle and displays the heterologous polypeptide on the surface thereof.

C-terminal display can also be useful for displaying heterologous polypeptides with respect to which a free C terminus is important for proper folding and/or retention of biological function. Examples of such polypeptides include PDZ domain proteins.

C-terminal display bypasses secretion problems encountered with N-terminal display systems. With N-terminal display, it is generally thought that the heterologous polypeptide on the N-terminus must pass through a pore-like structure in the periplasmic membrane in order to enter the periplasmic space with the C-terminus remaining as an anchor in the membrane. The fusion protein is then assembled into a phage particle from the membrane. Using C-terminal display, it is not necessary to have the fusion protein secrete into the host cell periplasm in order to assemble phage particles. C-terminal display is, therefore, useful to display any heterologous polypeptide and is particularly useful to display polypeptides which are difficult to display using N-terminal phage display techniques.

Other uses for C-terminal display are known in the art, for example as described in WO00/06717.

In some instances, simultaneous display of heterologous polypeptides on both the C and N termini is useful. For example, such display would obviate the inherent limitations of methods heretofore utilized in the art to obtain selectants exhibiting desired characteristics in screening/selection assays requiring the proximal display of two polypeptides, such as an enzyme and a reaction substrate/product, etc. See, e.g. Pedersen et al., Proc. Natl. Acad. Sci. USA, 95:10523-10528 (1998); Demartis et al., J. Mol. Biol. (1999), 286:617-633.

A heterologous DNA is preferably in the form of a replicable transcription or expression vector, such as a plasmid, phage or phagemid which can be constructed with relative ease and readily amplified. These vectors generally contain a promoter, a signal sequence, phenotypic selection genes, origins of replication, and other necessary components which are known to those of ordinary skill in this art. Construction of suitable vectors containing these components as well as the gene encoding one or more desired cloned polypeptides are prepared using standard recombinant DNA procedures as described in Sambrook et al., above. Isolated DNA fragments to be combined to form the vector are cleaved, tailored, and ligated together in a specific order and orientation to generate the desired vector.

The gene encoding the desired polypeptide (a fusion polypeptide of the invention) can be obtained by methods known in the art (see generally, Sambrook et al.). If the sequence of the gene is known, the DNA encoding the gene may be chemically synthesized (Merrfield, J. Am. Chem. Soc., 85:2149 (1963)). If the sequence of the gene is not known, or if the gene has not previously been isolated, it may be cloned from a cDNA library (made from RNA obtained from a suitable tissue in which the desired gene is expressed) or from a suitable genomic DNA library. The gene is then isolated using an appropriate probe. For cDNA libraries, suitable probes include monoclonal or polyclonal antibodies (provided that the cDNA library is an expression library), oligonucleotides, and complementary or homologous cDNAs or fragments thereof. The probes that may be used to isolate the gene of interest from genomic DNA libraries include cDNAs or fragments thereof that encode the same or a similar gene, homologous genomic DNAs or DNA fragments, and oligonucleotides. Screening the cDNA or genomic library with the selected probe is conducted using standard procedures as described in chapters 10-12 of Sambrook et al., above.

An alternative means to isolating the gene encoding the protein of interest is to use polymerase chain reaction methodology (PCR) as described in section 14 of Sambrook et al., above. This method requires the use of oligonucleotides that will hybridize to the gene of interest; thus, at least some of the DNA sequence for this gene must be known in order to generate the oligonucleotides.

After the gene has been isolated, it may be inserted into a suitable vector (preferably a plasmid) for amplification, as described generally in Sambrook et al.

The DNA is cleaved using the appropriate restriction enzyme or enzymes in a suitable buffer. In general, about 0.2-1 μg of plasmid or DNA fragments is used with about 1-2 units of the appropriate restriction enzyme in about 20 μl of buffer solution. Appropriate buffers, DNA concentrations, and incubation times and temperatures are specified by the manufacturers of the restriction enzymes. Generally, incubation times of about one or two hours at 37° C. are adequate, although several enzymes require higher temperatures. After incubation, the enzymes and other contaminants are removed by extraction of the digestion solution with a mixture of phenol and chloroform, and the DNA is recovered from the aqueous fraction by precipitation with ethanol or other DNA purification technique.

To ligate the DNA fragments together to form a functional vector, the ends of the DNA fragments must be compatible with each other. In some cases, the ends will be directly compatible after endonuclease digestion. However, it may be necessary to first convert the sticky ends commonly produced by endonuclease digestion to blunt ends to make them compatible for ligation. To blunt the ends, the DNA is treated in a suitable buffer for at least 15 minutes at 15° C. with 10 units of the Klenow fragment of DNA polymerase I (Klenow) in the presence of the four deoxynucleotide triphosphates. The DNA is then purified by phenol-chloroform extraction and ethanol precipitation or other DNA purification technique.

The cleaved DNA fragments may be size-separated and selected using DNA gel electrophoresis. The DNA may be electrophoresed through either an agarose or a polyacrylamide matrix. The selection of the matrix will depend on the size of the DNA fragments to be separated. After electrophoresis, the DNA is extracted from the matrix by electroelution, or, if low-melting agarose has been used as the matrix, by melting the agarose and extracting the DNA from it, as described in sections 6.30-6.33 of Sambrook et al., supra.

The DNA fragments that are to be ligated together (previously digested with the appropriate restriction enzymes such that the ends of each fragment to be ligated are compatible) are put in solution in about equimolar amounts. The solution will also contain ATP, ligase buffer and a ligase such as T4 DNA ligase at about 10 units per 0.5 μg of DNA. If the DNA fragment is to be ligated into a vector, the vector is at first linearized by cutting with the appropriate restriction endonuclease(s). The linearized vector is then treated with alkaline phosphatase or calf intestinal phosphatase. The phosphatasing prevents self-ligation of the vector during the ligation step.

After ligation, the vector with the foreign gene now inserted is purified as described above and transformed into a suitable host cell such as those described above by suitable techniques known in the art, for example by electroporation using known and commercially available electroporation instruments and the procedures outlined by the manufacturers and described generally in Dower et al., above.

Electroporation may be carried out using methods known in the art and described, for example, in U.S. Pat. No. 4,910,140; U.S. Pat. No. 5,186,800; U.S. Pat. No. 4,849,355; U.S. Pat. No. 5,173,158; U.S. Pat. No. 5,098,843; U.S. Pat. No. 5,422,272; U.S. Pat. No. 5,232,856; U.S. Pat. No. 5,283,194; U.S. Pat. No. 5,128,257; U.S. Pat. No. 5,750,373; U.S. Pat. No. 4,956,288 or any other known batch or continuous electroporation.

Typically, electrocompetent cells are mixed with a solution of DNA at the desired concentration at ice temperatures. An aliquot of the mixture is placed into a cuvette and placed in an electroporation instrument, e.g., GENE PULSER (Biorad) having a typical gap of 0.2 cm. Each cuvette is electroporated as described by the manufacturer. Typical settings are: voltage=2.5 kV, resistance=200 ohms, capacitance=25 mF. The cuvette is then immediately removed, SOC media (Maniatis) is added, and the sample is transferred to a 250 mL baffled flask. The contents of several cuvettes may be combined after electroporation. The culture is then shaken at 37° C. to culture the transformed cells.

The transformed cells are generally selected by growth on an antibiotic, commonly tetracycline (tet) or ampicillin (amp), to which they are rendered resistant due to the presence of tet and/or amp resistance genes in the vector.

After selection of the transformed cells, these cells are grown in culture and the vector DNA (plasmid or other vector with the foreign gene inserted) may then be isolated. Vector DNA can be isolated using methods known in the art. Two suitable methods are the small scale preparation of DNA and the large-scale preparation of DNA as described in sections 1.25-1.33 of Sambrook et al., supra. The isolated DNA can be purified by methods known in the art such as that described in section 1.40 of Sambrook et al., above and as described above. This purified DNA is then analyzed by restriction mapping and/or DNA sequencing. DNA sequencing is generally performed by either the method of Messing et al., Nucleic Acids Res., 9:309 (1981) or by the method of Maxam et al., Meth. Enzymol., 65:499 (1980).

As described above, this invention also contemplates fusing the gene encoding the desired polypeptide (gene 1) to a second gene (gene 2) such that a fusion protein is generated during transcription. Gene 2 is typically a variant coat protein gene of a filamentous phage, for example phage M13 or a related phage, and the gene is for example the coat protein VIII gene, or a fragment thereof. See U.S. Pat. No. 5,750,373; WO 95/34683. Fusion of genes 1 and 2 may be accomplished by inserting gene 2 into a particular site on a plasmid that contains gene 1, or by inserting gene 1 into a particular site on a plasmid that contains gene 2 using the standard techniques described above. Gene 1 can be fused to the N and/or C terminus of gene 2. In some embodiments, gene 2 has a heterologous gene fused simultaneously to it N and C termini. In one embodiment, the heterologous gene is the same on both termini. In one embodiment, the heterologous gene on one terminus is different/distinct from the heterologous gene on the other terminus.

In some embodiments, a fusion polypeptide may comprise between the first and second polypeptide sequences a third heterologous sequence, for example a molecular tag for identifying and/or capturing and purifying the transcribed fusion protein. For example, the third heterologous sequence may encode Herpes simplex virus glycoprotein D (Paborsky et al., 1990, Protein Engineering, 3:547-553) which can be used to affinity purify the fusion protein through binding to an anti-gD antibody. The third heterologous sequence may also code for a polyhistidine, e.g., (his)₆ (Sporeno et al., 1994, J. Biol. Chem., 269:10991-10995; Stuber et al., 1990, Immunol. Methods, 4:121-152, Waeber et al., 1993, FEBS Letters, 324:109-112), which can be used to identify and/or purify the fusion protein through binding to a metal ion (Ni) column (QIAEXPRESS Ni-NTA protein Purification System, Quiagen, Inc.). Other affinity tags known in the art may be used and encoded by the third heterologous sequence. Where there is bi-terminal fusion, and the fusion is to two different heterologous sequences, the C-terminal heterologous polypeptide can and is generally one that is not varied in sequence within a population comprising a plurality of sequences. For e.g., where a bi-terminal fusion polypeptide is used to display an enzyme and a substrate, and an objective is to obtain a variant of the enzyme exhibiting a desired characteristic, combinatorial mutation can be effected within the sequence encoding the enzyme, while the sequence encoding the substrate on the C-terminal end of the fusion polypeptide is kept invariant.

Insertion of a gene into a plasmid requires that the plasmid be cut at the precise location that the gene is to be inserted. Thus, there must be a restriction endonuclease site at this location (preferably a unique site such that the plasmid will only be cut at a single location during restriction endonuclease digestion). The plasmid is digested, phosphatased, and purified as described above. The gene is then inserted into this linearized plasmid by ligating the two DNAs together. Ligation can be accomplished if the ends of the plasmid are compatible with the ends of the gene to be inserted. If the restriction enzymes are used to cut the plasmid and isolate the gene to be inserted create blunt ends or compatible sticky ends, the DNAs can be ligated together directly using a ligase such as bacteriophage T4 DNA ligase and incubating the mixture at 16° C. for 14 hours in the presence of ATP and ligase buffer as described in section 1.68 of Sambrook et al., above. If the ends are not compatible, they must first be made blunt by using the Klenow fragment of DNA polymerase I or bacteriophage T4 DNA polymerase, both of which require the four deoxyribonucleotide triphosphates to fill-in overhanging single-stranded ends of the digested DNA. Alternatively, the ends may be blunted using a nuclease such as nuclease S1 or mung-bean nuclease, both of which function by cutting back the overhanging single strands of DNA. The DNA is then religated using a ligase as described above. In some cases, it may not be possible to blunt the ends of the gene to be inserted, as the reading frame of the coding region will be altered. To overcome this problem, oligonucleotide linkers may be used. The linkers serve as a bridge to connect the plasmid to the gene to be inserted. These linkers can be made synthetically as double stranded or single stranded DNA using standard methods. The linkers have one end that is compatible with the ends of the gene to be inserted; the linkers are first ligated to this gene using ligation methods described above. The other end of the linkers is designed to be compatible with the plasmid for ligation. In designing the linkers, care must be taken to not destroy the reading frame of the gene to be inserted or the reading frame of the gene contained on the plasmid. In some cases, it may be necessary to design the linkers such that they code for part of an amino acid, or such that they code for one or more amino acids.

Between gene 1 and gene 2, DNA encoding a termination codon may be inserted, such termination codons are UAG (amber), UAA (ocher) and UGA (opel). (Microbiology, Davis et al. Harper & Row, New York, 1980, pages 237, 245-47 and 274). The termination codon expressed in a wild type host cell results in the synthesis of the gene 1 protein product without the gene 2 protein attached. However, growth in a suppressor host cell results in the synthesis of detectable quantities of fused protein. Such suppressor host cells contain a tRNA modified to insert an amino acid in the termination codon position of the mRNA thereby resulting in production of detectable amounts of the fusion protein. Such suppressor host cells are well known and described, such as E. coli suppressor strain (Bullock et al., BioTechniques 5:376-379 [1987]). Any acceptable method may be used to place such a termination codon into the mRNA encoding the fusion polypeptide.

The suppressible codon may be inserted between the first gene encoding a polypeptide, and a second gene encoding at least a portion of a variant phage coat protein. Alternatively, the suppressible termination codon may be inserted adjacent to the fusion site by replacing the last amino acid triplet in the polypeptide or the first amino acid in the variant phage coat protein. When the plasmid containing the suppressible codon is grown in a suppressor host cell, it results in the detectable production of a fusion polypeptide containing the polypeptide and the coat protein. When the plasmid is grown in a non-suppressor host cell, the polypeptide is synthesized substantially without fusion to the phage coat protein due to termination at the inserted suppressible triplet encoding UAG, UAA, or UGA. In the non-suppressor cell the polypeptide is synthesized and secreted from the host cell due to the absence of the fused phage coat protein which otherwise anchored it to the host cell.

Gene 1 may encode a mammalian protein, e.g. a protein selected from human growth hormone (hGH), N-methionyl human growth hormone, bovine growth hormone, parathyroid hormone, thyroxine, insulin A-chain, insulin B-chain, proinsulin, relaxin A-chain, relaxin B-chain, prorelaxin, glycoprotein hormones such as follicle stimulating hormone (FSH), thyroid stimulating hormone (TSH), leutinizing hormone (LH), glycoprotein hormone receptors, calcitonin, glucagon, factor VIII, an antibody (full length or fragment thereof (e.g., Fab, F(ab′)₂, scFv), lung surfactant, urokinase, streptokinase, human tissue-type plasminogen activator (t-PA), bombesin, coagulation cascade factors including factor VII, factor IX, and factor X, thrombin, hemopoietic growth factor, tumor necrosis factor-alpha and -beta, enkephalinase, human serum albumin, mullerian-inhibiting substance, mouse gonadotropin-associated peptide, a microbial protein, such as betalactamase, tissue factor protein, inhibin, activin, vascular endothelial growth factor (VEGF), receptors for hormones or growth factors; integrin, thrombopoietin (TPO), protein A or D, rheumatoid factors, nerve growth factors such as NGF-alpha, platelet-growth factor, transforming growth factors (TGF) such as TGF-alpha and TGF-beta, insulin-like growth factor-I and -II, insulin-like growth factor binding proteins, CD-4, DNase, latency associated peptide, erythropoietin (EPO), osteoinductive factors, interferons such as interferon-alpha, -beta, and -gamma, colony stimulating factors (CSFs) such as M-CSF, GM-CSF, and G-CSF, interleukins (ILs) such as IL-1, IL-2, IL-3, IL-4, IL-6, IL-8, IL-10, IL-12, superoxide dismutase; decay accelerating factor, viral antigen, hepatocyte growth factor (HGF), c-met, HIV envelope proteins such as GP120, GP140, atrial natriuretic peptides A, B, or C, immunoglobulins, as well as variants, fragments of, and antibodies against any of the above-listed proteins.

A heterologous polypeptide portion of the fusion protein may contain as few as 4-10 or up to 20-30 amino acid residues and even up to about 50-80 residues. These smaller peptides are useful in determining the antigenic properties of the peptides, in mapping the antigenic sites of proteins, etc. A heterologous polypeptide may also contain one or more subunits containing at least about 100 amino acid residues which may be folded to form a plurality of rigid secondary structures displaying a plurality of amino acids capable of interacting with the target. If a heterologous polypeptide portion of the fusion protein is mutated to form a library and subjected to phage display selection the polypeptide can be mutated at codons corresponding to the amino acids capable of interacting with the target so that the integrity of the rigid secondary structures will be preserved. The residues can be determined by alanine scanning mutagenesis, for example. U.S. Pat. No. 5,580,723 and U.S. Pat. No. 5,766,854.

Phage display of proteins, peptides and mutated variants thereof, including constructing a family of variant replicable vectors containing a transcription regulatory element operably linked to a gene fusion encoding a fusion polypeptide, transforming suitable host cells, culturing the transformed cells to form phage particles which display the fusion polypeptide on the surface of the phage particle, contacting the recombinant phage particles with a target molecule so that at least a portion of the particles exhibit a selection characteristic (e.g., binding to a target), separating the particles which exhibit a desired characteristic from those that do not are known and may be used in methods and compositions of the invention. See U.S. Pat. No. 5,750,373; WO 97/09446; U.S. Pat. No. 5,514,548; U.S. Pat. No. 5,498,538; U.S. Pat. No. 5,516,637; U.S. Pat. No. 5,432,018; WO 96/22393; U.S. Pat. No. 5,658,727; U.S. Pat. No. 5,627,024; WO 97/29185; O'Boyle et al, 1997, Virology, 236:338-347; Soumillion et al, 1994, Appl. Biochem. Biotech., 47:175-190; O'Neil and Hoess, 1995, Curr. Opin. Struct. Biol., 5:443-449; Makowski, 1993, Gene, 128:5-11; Dunn, 1996, Curr. Opin. Struct. Biol., 7:547-553; Choo and Klug, 1995, Curr. Opin. Struct. Biol., 6:431-436; Bradbury and Cattaneo, 1995, TINS, 18:242-249; Cortese et al., 1995, Curr. Opin. Struct. Biol., 6:73-80; Allen et al., 1995, TIBS, 20:509-516; Lindquist and Naderi, 1995, FEMS Micro. Rev., 17:33-39; Clarkson and Wells, 1994, Tibtech, 12:173-184; Barbas, 1993, Curr. Opin. Biol., 4:526-530; McGregor, 1996, Mol. Biotech., 6:155-162; Cortese et al., 1996, Curr. Opin. Biol., 7:616-621; McLafferty et al., 1993, Gene, 128:29-36.

In one example, gene 1 encodes the light chain or the heavy chain of an antibody or fragments thereof, such as Fab, F(ab′)₂, Fv, diabodies, linear antibodies, etc. Gene 1 may also encode a single chain antibody (scFv). The preparation of libraries of antibodies or fragments thereof is well known in the art and any of the known methods may be used to construct a family of transformation vectors which may be transformed into host cells using the method of the invention. Libraries of antibody light and heavy chains in phage (Huse et al, 1989, Science, 246:1275) and as fusion proteins in phage or phagemid are well known and can be prepared according to known procedures. See, e.g., Vaughan et al., Barbas et al., Marks et al., Hoogenboom et al., Griffiths et al., de Kruif et al., noted above, and WO00/06717; WO 98/05344; WO 98/15833; WO 97/47314; WO 97/44491; WO 97/35196; WO 95/34648; U.S. Pat. No. 5,712,089; U.S. Pat. No. 5,702,892; U.S. Pat. No. 5,427,908; U.S. Pat. No. 5,403,484; U.S. Pat. No. 5,432,018; U.S. Pat. No. 5,270,170; WO 92/06176; U.S. Pat. No. 5,702,892. Reviews have also been published. Hoogenboom, 1997, Tibtech, 15:62-70; Neri et al., 1995, Cell Biophysics, 27:47; Winter et al., 1994, Annu. Rev. Immunol., 12:433-455; Soderlind et al., 1992, Immunol. Rev., 130:109-124; Jefferies, 1998, Parasitology, 14:202-206.

Specific antibodies contemplated as being encoded by gene 1 include antibodies which bind to human leukocyte surface markers, cytokines and cytokine receptors, enzymes, etc. Specific leukocyte surface markers include CD1a-c, CD2, CD2R, CD3-CD10, CD11a-c, CDw12, CD13, CD14, CD15, CD15s, CD16, CD16b, CDw17, CD18-C41, CD42a-d, CD43, CD44, CD44R, CD45, CD45A, CD45B, CD45O, CD46-CD48, CD49a-f, CD50-CD51, CD52, CD53-CD59, CDw60, CD61, CD62E, CD62L, CD62P, CD63, CD64, CDw65, CD66a-e, CD68-CD74, CDw75, CDw76, CD77, CDw78, CD79a-b, CD80-CD83, CDw84, CD85-CD89, CDw90, CD91, CDw92, CD93-CD98, CD99, CD99R, CD100, CDw101, CD102-CD106, CD107a-b, CDw108, CDw109, CD115, CDw116, CD117, CD119, CD120a-b, CD121a-b, CD122, CDw124, CD126-CD129, and CD130. Other antibody binding targets include cytokines and cytokine superfamily receptors, hematopoietic growth factor superfamily receptors and preferably the extracellular domains thereof, which are a group of closely related glycoprotein cell surface receptors that share considerable homology including frequently a WSXWS domain and are generally classified as members of the cytokine receptor superfamily (see e.g. Nicola et al., Cell, 67:14 (1991) and Skoda, R. C. et al. EMBO J. 12:2645-2653 (1993)). Generally, these targets are receptors for interleukins (IL) or colony-stimulating factors (CSF). Members of the superfamily include, but are not limited to, receptors for: IL-2 (b and g chains) (Hatakeyama et al., Science, 244:551-556 (1989); Takeshita et al., Science, 257:379-382 (1991)), IL-3 (Itoh et al., Science, 247:324-328 (1990); Gorman et al., Proc. Natl. Acad. Sci. USA, 87:5459-5463 (1990); Kitamura et al., Cell, 66:1165-1174 (1991a); Kitamura et al., Proc. Natl. Acad. Sci. USA, 88:5082-5086 (1991b)), IL-4 (Mosley et al., Cell, 59:335-348 (1989), IL-5 (Takaki et al., EMBO J., 9:4367-4374 (1990); Tavernier et al., Cell, 66:1175-1184 (1991)), IL-6 (Yamasaki et al., Science, 241:825-828 (1988); Hibi et al., Cell, 63:1149-1157 (1990)), IL-7 (Goodwin et al., Cell, 60:941-951 (1990)), IL-9 (Renault et al., Proc. Natl. Acad. Sci. USA, 89:5690-5694 (1992)), granulocyte-macrophage colony-stimulating factor (GM-CSF) (Gearing et al., EMBO J., 8:3667-3676 (1991); Hayashida et al., Proc. Natl. Acad. Sci. USA, 244:9655-9659 (1990)), granulocyte colony-stimulating factor (G-CSF) (Fukunaga et al., Cell, 61:341-350 (1990a); Fukunaga et al., Proc. Natl. Acad. Sci. USA, 87:8702-8706 (1990b); Larsen et al., J. Exp. Med., 172:1559-1570 (1990)), EPO (D'Andrea et al., Cell, 57:277-285 (1989); Jones et al., Blood, 76:31-35 (1990)), Leukemia inhibitory factor (LIF) (Gearing et al., EMBO J., 10:2839-2848 (1991)), oncostatin M (OSM) (Rose et al., Proc. Natl. Acad. Sci. USA, 88:8641-8645 (1991)) and also receptors for prolactin (Boutin et al., Proc. Natl. Acad. Sci. USA, 88:7744-7748 (1988); Edery et al., Proc. Natl. Acad. Sci. USA, 86:2112-2116 (1989)), growth hormone (GH) (Leung et al., Nature, 330:537-543 (1987)), ciliary neurotrophic factor (CNTF) (Davis et al., Science, 253:59-63 (1991) and c-Mpl (M. Souyri et al., Cell 63:1137 (1990); I. Vigon et al., Proc. Natl. Acad. Sci. 89:5640 (1992)). Still other targets for antibodies made by the invention are erb2, erb3, erb4, IL-10, IL-12, IL-13, IL-15, vascular endothelial cell growth factor (VEGF), hepatocyte growth factor (HGF), c-met, etc.

Gene 1, encoding a desired polypeptide, may be altered at one or more selected codons. An alteration is defined as a substitution, deletion, or insertion of one or more codons in the gene encoding the polypeptide that results in a change in the amino acid sequence of the polypeptide as compared with the unaltered or native sequence of the same polypeptide. Preferably, the alterations will be by substitution of at least one amino acid with any other amino acid in one or more regions of the molecule. The alterations may be produced by a variety of methods known in the art. These methods include but are not limited to oligonucleotide-mediated mutagenesis and cassette mutagenesis.

In one embodiment, oligonucleotide-mediated mutagenesis is employed for preparing substitution, deletion, and insertion variants of gene 1. This technique is well known in the art as described by Zoller et al., Nucleic Acids Res., 10: 6487-6504 (1987). Briefly, gene 1 is altered by hybridizing an oligonucleotide encoding the desired mutation to a DNA template, where the template is the single-stranded form of the plasmid containing the unaltered or native DNA sequence of gene 1. After hybridization, a DNA polymerase is used to synthesize an entire second complementary strand of the template will thus incorporate the oligonucleotide primer, and will code for the selected alteration in gene 1.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75: 5765 (1978).

The DNA template is generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153: 3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with 32-Phosphate to identify the bacterial colonies that contain the mutated DNA.

The method described immediately above may be modified such that a homoduplex molecule is created wherein both strands of the plasmid contain the mutation(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thio-deoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101, as described above.

Mutants with more than one amino acid to be substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

Cassette mutagenesis is also a preferred method for preparing substitution, deletion, and insertion variants of gene 1. The method is based on that described by Wells et al., Gene, 34:315 (1985). The starting material is the plasmid (or other vector) comprising gene 1, the gene to be mutated. The codon(s) in gene 1 to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in gene 1. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. This plasmid now contains the mutated DNA sequence of gene 1.

In one embodiment, gene 1 is linked to gene 2 encoding at least a portion of a variant phage coat protein. Examples of coat protein genes include the genes encoding coat protein 8 of filamentous phage specific for E. coli, such as M13, f1 and fd phage. Transfection of host cells containing a replicable expression vector which encodes the gene fusion of gene 1 and gene 2 and production of phage particles according to standard procedures provides phage particles in which the polypeptide encoded by gene 1 is displayed on the surface of the phage particle.

Any number of host cells can be used in the present invention. For example, any cells which can be transformed (e.g., by electroporation) may be used as host cells in the present invention. Prokaryotes are suitable host cells for the invention. Suitable bacterial cells include E. coli (Dower et al., above; Taketo, Biochim. Biophys. Acta, (1988), 149:318), L. casei (Chassy and Flickinger, FEMS Microbiol. Lett., (1987), 44:173), Strept. lactis (Powell et al., Appl. Environ. Microbiol., (1988), 54:655; Harlander, Streptococcal Genetics, ed. J. Ferretti and R. Curtiss, III), page 229, American Society for Microbiology, Washington, D.C., (1987)), Strept. thermophilus (Somkuti and Steinberg, Proc. 4th Eur. Cong. Biotechnology, 1987, 1:412); Campylobacter jejuni (Miller et al., Proc. Natl. Acad. Sci., USA, (1988) 85:856), and other bacterial strains (Fielder and Wirth, Anal. Biochem., (1988), 170:38) including bacilli such as Bacillus subtilis, other enterobacteriaceae such as Salmonella typhimurium or Serratia marcesans, and various Pseudomonas species which may all be used as hosts. Other examples of suitable E. coli strains include JM101, E. coli K12 strain 294 (ATCC number 31,446), E. coli strain W3110 (ATCC number 27,325), E. coli X1776 (ATCC number 31,537), E. coli XL-1Blue (Stratagene), and E. coli B; however many other strains of E. coli, such as XL1-Blue MRF′, SURE, ABLE C, ABLE K, WM1100, MC1061, HB101, CJ136, MV1190, JS4, JS5, NM522, NM538, NM539, TG1 and many other species and genera of prokaryotes may be used as well. An example of a particularly suitable host cell is E. coli strain SS320, which was prepared by mating MC1061 cells with XL1-BLUE cells under conditions sufficient to transfer the fertility episome (F′ plasmid) of XL1-BLUE into the MC1061 cells. In general, mixing cultures of the two cell types and growing the mixture in culture medium for about one hour at 37° C. is sufficient to allow mating and episome transfer to occur. The new resulting E. coli strain has the genotype of MC1061 which carries a streptomycin resistance chromosomal marker and the genotype of the F′ plasmid which confers tetracycline resistance. The progeny of this mating is resistant to both antibiotics and can be selectively grown in the presence of streptomycin and tetracycline. Strain SS320 has been deposited with the American Type Culture Collection (ATCC), 10801 University Boulevard, Manassas, Va., USA on Jun. 18, 1998 and assigned Deposit Accession No. 98795.

The transformed cells are generally selected by growth on an antibiotic, commonly tetracycline (tet) or ampicillin (amp), to which they are rendered resistant due to the presence of tet and/or amp resistance genes in the vector.

Suitable phage and phagemid vectors for use in this invention include all known vectors for phage display. Additional examples include pComb8 (Gram, H., Marconi, L. A., Barbas, C. F., Collet, T. A., Lerner, R. A., and Kang, A. S. (1992) Proc. Natl. Acad. Sci. USA 89:3576-3580); pC89 (Felici, F., Catagnoli, L., Musacchio, A., Jappelli, R., and Cesareni, G. (1991) J. Mol. Biol. 222:310-310); pIF4 (Bianchi, E., Folgori, A., Wallace, A., Nicotra, M., Acali, S., Phalipon, A., Barbato, G., Bazzo, R., Cortese, R., Felici, F., and Pessi, A. (1995) J. Mol. Biol. 247:154-160); PM48, PM52, and PM54 (Iannolo, G., Minenkova, O., Petruzzelli, R., and Cesareni, G. (1995) J. Mol. Biol, 248:835-844); fdH (Greenwood, J., Willis, A. E., and Perham, R. N. (1991) J. Mol. Biol, 220:821-827); pfd8SHU, pfd8SU, pfd8SY, and fdISPLAY8 (Malik, P. and Perham, R. N. (1996) Gene, 171:49-51); “88” (Smith, G. P. (1993) Gene, 128:1-2); f88.4 (Zhong, G., Smith, G. P., Berry, J. and Brunham, R. C. (1994) J. Biol. Chem, 269:24183-24188); p8V5 (Affymax); MB1, MB20, MB26, MB27, MB28, MB42, MB48, MB49, MB56: Markland, W., Roberts, B. L., Saxena, M. J., Guterman, S. K., and Ladner, R. C. (1991) Gene, 109:13-19). Similarly, any known helper phage may be used when a phagemid vector is employed in the phage display system. Examples of suitable helper phage include M13-KO7 (Pharmacia), M13-VCS (Stratagene), and R408 (Stratagene).

After selection of the transformed cells, these cells are grown in culture and the vector DNA may then be isolated. Phage or phagemid vector DNA can be isolated using methods known in the art, for example, as described in Sambrook et al., Molecular Cloning: A Laboratory Manual, 2nd edition, (1989) Cold Spring Harbor Laboratory Press, Cold Spring Harbor, N.Y.

The isolated DNA can be purified by methods known in the art such as that described in section 1.40 of Sambrook et al., above and as described above. This purified DNA can then be analyzed by DNA sequencing. DNA sequencing may be performed by the method of Messing et al., Nucleic Acids Res., 9:309 (1981), the method of Maxam et al., Meth. Enzymol., 65:499 (1980), or by any other known method.

U.S. Pat. No. 5,750,373 and WO00/06717 describe generally how to produce and recover a product polypeptide by culturing a host cell transformed with a replicable expression vector (e.g., a phagemid) encoding the polypeptide.

The expression of polypeptides on the surface of bacteriophage has been developed and refined over several years. In particular, systems have been developed for displaying recombinant peptides, proteins, antigens and antibodies on the surface of filamentous bacteriophage. A number of filamentous phage have been identified which are able to infect gram negative bacteria, such as E. coli.

Fusion proteins containing variants of the major coat protein of any bacteriophage which is suitable for use in a known phage display system are within the scope of the present invention. Class I and class II filamentous phage are included within the scope of the invention. Class I includes strains Ff, IKe and If1; class II includes strains Pf1, Pf3 and Xf. The Ff phage include the virtually identical strains fd, f1 and M13.

The sequences of several known mature major coat proteins of filamentous bacteriophage aligned with the mature M13 coat protein VIII are shown in the Table below. Segments of the coat proteins were aligned with M13 protein VIII so as to provide maximum identity with the M13 protein without the introduction of any deletions or insertions. Numbering above the sequences refers to the residues of mature M13 protein VIII. Protein sequences are taken from the Dayhoff protein database (accession numbers: M13, COAB_BPFD; F1, COAB_BPFD; Fd, COAB_BPFD; Zj-2, COAB_BPZJ2; If-1, COAT_BPIF1; I2-2, COAB_BPI22; Ike, COAB_BPIKE). Homologous residues are indicated with dashes. A sequence having a single deletion is also known (WO 92/18619). It can be seen that there is considerable homology among the sequences of these coat proteins, particularly among the M13, f1, fd and Zj-2 coat proteins and among the If1, I22 and Ike coat proteins.

TABLE 1

1 2 3 4 5 6 7 8 9 10 11 12 13 14 15 16 17 18 19 20 21 22 23 24 25

| | |
|---|---|
| M13 | A E G D D P A K A A F N S L Q A S A T E Y I G Y A |
| Fl | - - - - - - - - - - - D - - - - - - - - - - - - - |
| Fd | - - - - - - - - - - - D - - - - - - - - - - - - - |
| Zj-2 | - - - - - - - - - - - D - - - - - - - - - - - - - |
| Ifl | D D A T S Q - - - - - D - - T - Q - - - M S - - - |
| I2-2 | S T A T S Y - T E - M - - - K T Q - - D L - D Q T |
| Ike | N A A T N Y - T E - M D - - K T Q - I D L - S Q T |

26 27 28 29 30 31 32 33 34 35 36 37 38 39 40 41 42 43 44 45 46 47 48 49 50

| | |
|---|---|
| M13 | W A M V V V I V G A T I G I K L F K K F T S K A S |
| Fl | - - - - - - - - - - - - - - - - - - - - - - - - - |
| Fd | - - - - - - - - - - - - - - - - - - - - - - - - - |
| Zj-2 | - - - - - - - - - - - - - - - - - - - - A - - - - |
| Ifl | - - L - - L V - - - - V - - - - - - - - V - R - - |
| I2-2 | - P V - T S V A V - G L A - R - - - - - S - - - V |
| Ike | - P V - T T V - V - G L V - R - - - - - S - - - V |

(SEQ ID NOS._-_)

All patent and literature references cited herein are incorporated herein by reference in their entirety.

Having generally described the invention, the same will be more readily understood by reference to the following examples, which are provided by way of illustration and are not intended as limiting.

### EXAMPLES

### Materials and Methods

Materials

E. coli XL1-Blue was from Stratagene. Enzymes and M13-K07 were from New England Biolabs. MaxiSorp immunoplates were from NUNC (Roskilde, Denmark). Bovine serum albumin (BSA) and Tween 20 were from Sigma. Horse radish peroxidase/anti-M13 antibody conjugate was from Pharmacia Biotech. 3,3′,5,5′-Tetramethyl-benzidine/H2O2 (TMB) peroxidase was from Kirkegaard & Perry Laboratories Inc. Western blotting reagents were from Invitrogen. PVDF filters were from Bio-Rad. Horseradish peroxidase/rabbit anti-mouse conjugate was from Jackson ImmunoResearch Laboratories.

Library Construction

A previously described phagemid selected for C-terminal display (pS1403a) (16) was modified by replacing the polyHis tag at the C-terminus of P8 with a peptide sequence (RAAERWDTWV) selected for binding to the Erbin PDZ domain (17). The resulting phagemid was designated pS1403aEL.

First generation libraries were constructed as described (15; 29; 30) with “stop template” versions of pS1403aEL that contained stop codons inserted in the regions to be mutagenized. For each library, the Kunkel mutagenesis method (31) was used with a mutagenic oligonucleotide designed to simultaneously repair the stop codons and introduce NNS (N=A/G/C/T, S=G/C) degenerate codons at the desired sites. A total of eight libraries were constructed and each library contained ˜10¹⁰ unique members. Libraries 1 through 8 mutagenized positions 1-6, 7-14, 15-22, 23-30, 31-38, 39-46, 47-53, or 54-60, respectively. The second generation library was constructed similarly with a stop template version of the phagemid encoding for the variant P8-C-S14 and a mutagenic oligonucleotide that replaced codons with tailored degenerate codons (FIG. 6A). The second generation library contained 4×10¹⁰ unique members.

Library Sorting and Analysis

Phage from the libraries described above were cycled through rounds of binding selection, as described previously (30), with a GST-Erbin PDZ domain fusion protein (32) coated on 96-well Maxisorp immunoplates as the capture target. Phage were propagated in E. coli XL1-blue in medium supplemented with M13-KO7 helper phage to facilitate phage production. For the first generation libraries, the medium was also supplemented with 10∝M IPTG to induce expression of the library.

Individual clones from the selected pools were grown in a 96-well format in 400 μL of 2YT broth supplemented with carbenicillin and helper phage. The culture supernatants were used in phage ELISAs with plates coated with the GST-Erbin PDZ domain fusion protein. Positive clones that exhibited strong ELISA signals were subjected to DNA sequence analysis (15).

Sequences from the first generation libraries were analyzed with the program SGCOUNT (33). SGCOUNT aligned each DNA sequence against the wt DNA sequence by using a Needleman-Wunch pairwise alignment algorithm, translated each aligned sequence of acceptable quality, and tabulated the occurrence of each natural amino acid at each position. The tabulated data were normalized for bias in the degenerate NNS codon (e.g. the NNS codon contains three unique codons for Arg, and thus, the Arg occurrence was divided by three). The normalized data were used to calculate the percent occurrence of each amino acid at each position (Table 2). The following number of unique clones were analyzed for libraries 1 through 8, respectively: 86, 90, 74, 68, 90, 41, 92, and 78.

The per residue variation was estimated using the Shannon entropy as a measure of diversity (19). Shannon Entropy is defined for protein sites by the formula
H=−Σᵢ₌₁₋₂₀ pᵢ log₂ pᵢ

where pᵢ is the fraction of residues at the site that are of type i.

Site-Directed Mutagenesis

Mutagenesis was performed using the method of Kunkel et al (31).

Phage ELISA for Measuring Display Enhancement

Phage ELISA protocols were adapted from previous work (15; 34). Cultures of E. coli XL1-Blue harboring phagemids were grown for 2 hours at 37° C. in 1 ml of 2YT supplemented with 50 μg/mL carbenicillin, 5 μg/ml tetracycline, and M13-KO7 helper phage (10¹⁰ phage/ml). Kanamycin was added to a final concentration of 25 μg/mL and the cultures were incubated for a further 6 hours. The cultures were transferred to 25 ml of 2YT, 50 μg/mL carbenicillin, 25 μg/mL kanamycin, 25 μM IPTG and grown overnight at 37° C. Phage were harvested from the culture supernatant by precipitating twice with PEG/NaCl (30) and resuspended in 1.0 ml of 10 mM Tris, 1 mM EDTA, pH 7.6. Phage concentrations were determined spectrophotometrically (ε₂₆₈=1.2×10⁸ M⁻¹ cm⁻¹).

Maxisorp immunoplates were coated with the GST-Erbin PDZ domain fusion protein overnight at 4° C., blocked for 1 hour at room temperature with 0.5% BSA in PBS and washed five times with PBS, 0.05% Tween 20. Phage particles were diluted serially into ELISA buffer (PBS, 0.5% BSA, 0.1% Tween 20) and 100 μL were transferred to coated wells. After 1 hour, plates were washed 15 times with PBS, 0.05% Tween 20, incubated with 100 μL of 1:5000 (v/v) horse radish peroxidase/anti-M13 antibody conjugate in ELISA buffer for 30 minutes, and then washed eight times with PBS, 0.05% Tween 20 and twice with PBS. Plates were developed using a TMB peroxidase substrate system (100 μL), quenched with 1.0 M H₃PO₄ (100 μL) and read spectrophotometrically at 450 nm.

Western Blotting

Phage samples were purified as described above and denatured at 95° C. for 2 min in SDS-PAGE sample buffer (2% SDS, 20 mM Tris, pH 6.8, 10% glycerol). The denatured samples were run on a 18% Tris-glycine gel and then electrotransferred to a PVDF filter. The PVDF filter was blocked with 2% milk and 0.1% Tween 20 in 20 mM Tris, pH 7.5, 0.15 M NaCl (blocking buffer) overnight at 4° C. The filter was incubated with 3 nM anti-gDtag antibody in blocking buffer for 1 hour at room temperature. The filter was washed with PBS, 0.05% Tween 20, incubated with horseradish peroxidase/rabbit anti-mouse Fab conjugate (1:10,000 dilution) for 1 hour and visualized with ECL™ Western blotting detection reagent.

Results

A previously described P8 moiety (P8-C) was used in a phagemid-based display system (16). P8-C consists of the 50-residue mature P8 sequence followed by a 10-residue C-terminal linker that was selected for efficient peptide display (Table 2). The C terminus of P8-C was modified by the addition of a peptide (Erbin ligand) that binds to the Erbin PDZ domain (17). In this system, the level of Erbin ligand display could be used as an indicator of the incorporation efficiency of the P8-C fusion protein into a phage coat composed of wt P8 supplied by a helper phage.

TABLE 2
Database of P8-C sequence diversity compatible with C-terminal display

| | Percent Occurrence | | | | | | | | | | | | | | | | | | | |
|---|---|---|---|---|---|---|---|---|---|---|---|---|---|---|---|---|---|---|---|---|
| wt | P | G | A | C | V | L | I | M | F | Y | W | S | T | N | Q | D | E | H | K | R |
| A1 | 1 | 5 | 16 | 7 | 4 | 1 | 7 | 3 | 5 | 12 | | 8 | 7 | 3 | | 7 | 10 | 2 | | 2 |
| E2 | 3 | 8 | 13 | | 8 | 2 | | 3 | | 3 | 2 | 3 | | | 6 | 21 | 25 | 2 | 3 | 1 |
| G3 | 6 | 24 | 8 | 2 | 7 | 6 | 2 | | 4 | | 2 | 11 | 4 | 2 | 2 | 6 | 10 | | | 3 |
| D4 | 5 | 8 | 4 | 2 | 8 | 2 | 2 | 3 | | | | 4 | 1 | 3 | 2 | 36 | 14 | 8 | | 1 |
| D5 | 2 | 5 | 2 | | 2 | 3 | | 6 | 5 | 6 | 3 | 5 | 4 | 3 | | 30 | 17 | 8 | | |
| P6 | 20 | 2 | 3 | 3 | | | | | 6 | 14 | | 5 | 3 | 3 | 2 | 25 | 2 | 11 | | 1 |
| A7 | | 42 | 37 | 7 | 1 | 1 | 2 | 2 | 2 | | 4 | 1 | 1 | | | | | | | |
| K8 | | 2 | 7 | | 20 | 19 | 20 | 9 | 2 | | | 2 | 2 | | 2 | | | 4 | 4 | 4 |
| A9 | | 2 | 19 | 2 | 10 | 11 | 4 | 8 | | 2 | 4 | 1 | 2 | 2 | 16 | 4 | 8 | | | 4 |
| A10 | | 3 | 94 | | | | | | | 2 | | | | | | | | | | |
| F11 | | | | | | | | 6 | 21 | | 72 | | | | | | | | | |
| N12 | | 1 | 17 | | 3 | 4 | 6 | 10 | 2 | | | 3 | 1 | 6 | 12 | | 12 | 4 | 6 | 15 |
| S13 | | 2 | 15 | | 5 | 6 | | 8 | 2 | 4 | 2 | 17 | 3 | | 8 | 4 | 19 | 4 | | |
| L14 | | | | | 12 | 63 | | 26 | | | | | | | | | | | | |
| Q15 | | 1 | 2 | | 10 | 26 | 9 | 4 | 11 | 13 | 2 | 1 | 6 | | 7 | | 7 | | | |
| A16 | | 6 | 11 | 2 | 6 | 4 | 2 | 4 | 2 | | | 7 | 10 | | 7 | 9 | 18 | 7 | 2 | 3 |
| S17 | 2 | | 4 | 4 | 9 | 12 | | 10 | 6 | 6 | 6 | 6 | 5 | 6 | 6 | 6 | 8 | | 2 | 1 |
| A18 | 1 | 3 | 18 | 16 | 26 | 4 | 13 | 11 | | | | 5 | 1 | | | | 2 | | | |
| T19 | 2 | 6 | 6 | 15 | 11 | 8 | 2 | 2 | 2 | 4 | 2 | 5 | 4 | 6 | 4 | 4 | 10 | | 2 | 3 |
| E20 | 1 | 5 | 5 | 5 | 5 | 2 | 2 | | 2 | 2 | | 6 | 3 | | 9 | 14 | 31 | 2 | 4 | 3 |
| Y21 | | | 5 | 7 | 12 | 7 | 14 | 14 | 11 | 5 | 14 | 2 | 2 | | 2 | | 2 | 2 | 2 | |
| I22 | | 2 | 7 | 17 | 36 | 3 | 25 | 2 | | | | 2 | 4 | | | | | | | 2 |
| G23 | | 12 | 12 | 15 | 7 | 7 | | 12 | 5 | 10 | 2 | 6 | 2 | | 2 | | 5 | | 2 | 2 |
| Y24 | | 2 | 4 | 8 | 2 | 15 | 4 | 11 | 4 | 6 | 15 | 3 | | 4 | 2 | 8 | 13 | | | |
| A25 | | 27 | 67 | | | | | | | | | 3 | 3 | | | | | | | |
| W26 | | | | | | | | 3 | 6 | | 91 | | | | | | | | | |
| A27 | | 8 | 36 | | 15 | 4 | | 3 | | | | 6 | 4 | | 11 | | 8 | 5 | | |
| M28 | | | | | 1 | 27 | 7 | 57 | 2 | 3 | 2 | | 1 | | | | | | | |
| V29 | | | 7 | 11 | 52 | 18 | 3 | 6 | 3 | | | | | | | | | | | |
| V30 | | | 4 | 10 | 43 | 11 | 15 | 3 | 13 | | | 2 | | | | | | | | |
| V31 | | 3 | 10 | 23 | 25 | 24 | | | | | | 7 | 6 | | | 2 | | | | |
| I32 | | | | 19 | 39 | 12 | 21 | 2 | | | 4 | 1 | 3 | | | | | | | |
| V33 | | 3 | 8 | 20 | 25 | | 3 | 5 | | 3 | 12 | 6 | 8 | 2 | 3 | | | | | |
| G34 | | 16 | 1 | 22 | 13 | 10 | | 10 | | | | 10 | 16 | | | | | | | |
| A35 | | 21 | 42 | 21 | 2 | 1 | | 2 | 4 | | | 4 | 4 | | | | | | | |
| T36 | | 4 | 11 | 20 | 15 | 10 | 2 | 7 | 6 | 6 | | 6 | 7 | | 2 | 2 | 2 | | | 1 |
| I37 | | 2 | 12 | 20 | 20 | 1 | 8 | 3 | 8 | 2 | 5 | 4 | 9 | | 2 | | 2 | 2 | | 1 |
| G38 | | 40 | | 21 | 2 | 13 | | 4 | | | | 4 | 13 | 2 | | | | | | |
| I39 | | | | | 3 | 16 | 19 | 26 | 3 | | 32 | | | | | | | | | |
| K40 | | 2 | | | | | | | | | | | 4 | | | | | | 57 | 36 |
| L41 | | | | | 3 | 17 | | 3 | 3 | | 73 | | | | | | | | | |
| F42 | | 2 | 5 | 6 | 19 | 4 | 15 | 6 | 22 | 3 | 19 | | | | | | | | | |
| K43 | | 2 | 2 | 4 | 2 | 4 | 11 | 4 | | | 7 | 1 | | | | | | | 46 | 20 |
| K44 | | 8 | | | 3 | 2 | | | 3 | | 16 | 2 | 3 | 6 | 6 | 3 | 3 | | 32 | 10 |
| F45 | | 1 | 3 | | 4 | 5 | | 8 | 24 | 5 | 47 | | | | 3 | | | | | |
| T46 | | 4 | 14 | 4 | 16 | 4 | 4 | 11 | 18 | 4 | 11 | 4 | 11 | | | | | | | |
| S47 | | 20 | 5 | 9 | 11 | 6 | 5 | 7 | 4 | 5 | 9 | 4 | | | 2 | 2 | 2 | 2 | 2 | 7 |
| K48 | 2 | 17 | 3 | 6 | 4 | 4 | | 14 | 10 | 6 | 13 | 6 | 1 | 2 | 3 | 3 | 5 | | 0 | 2 |
| A49 | 3 | 20 | 9 | 3 | 7 | 1 | 3 | 9 | 10 | | 7 | 6 | 3 | 3 | 3 | 2 | 2 | 2 | 3 | 3 |
| S50 | 2 | 26 | 12 | 4 | 6 | 9 | 6 | 2 | 2 | 2 | 2 | 7 | 3 | | 4 | 4 | 6 | 2 | 2 | 1 |
| A51 | 12 | 17 | 11 | 4 | 6 | 7 | | | | 8 | 6 | 9 | 3 | | 4 | 2 | 2 | 6 | 2 | 2 |
| W52 | 8 | 13 | 5 | 9 | 7 | 3 | 2 | 7 | | 3 | 7 | 5 | 3 | 7 | 5 | 2 | 7 | 3 | | 5 |
| E53 | 1 | 12 | 5 | 4 | 7 | 4 | | 7 | | | | 7 | 6 | 2 | 7 | 2 | 25 | | 7 | 5 |
| E54 | | 26 | 27 | 4 | 11 | | | | | | | 7 | | | 7 | 4 | 11 | | | 1 |
| N55 | 4 | 36 | 5 | | 7 | 1 | | 2 | | 2 | | 8 | 5 | 4 | 2 | 9 | 9 | | 2 | 1 |
| I56 | 1 | 10 | 2 | | 3 | | 1 | 1 | | | | 2 | 1 | 1 | 6 | 43 | 21 | 3 | 1 | |
| D57 | 10 | 13 | 3 | | 18 | 6 | | 3 | 3 | | | 32 | 3 | 3 | | 0 | | | | 6 |
| S58 | 3 | 10 | 1 | | 7 | 1 | | 4 | | | | 9 | 6 | | 2 | 2 | 2 | 2 | 47 | 4 |
| A59 | 3 | 26 | 3 | | | | | | | | | 9 | 25 | | 4 | 15 | 9 | 2 | | 3 |
| P60 | 8 | 8 | 5 | 8 | | 1 | 3 | | 3 | | | 37 | 4 | 5 | 3 | 8 | 5 | 3 | | 1 |

The percent occurrence of each amino acid type at each position in the P8-C molecule was calculated after normalization for codon bias. The percent occurrence of wt at each position is shown in bold text. Positions 1-50 correspond to the wt P8 sequence, while positions 51-60 correspond to a linker sequence. The data were obtained from DNA sequencing of library clones after two rounds of selection for C-terminal peptide display. See Materials and Methods for further details.

Database of P8-C Diversity Compatible with C-Terminal Display

A comprehensive assessment was compiled of the sequence diversity that could be tolerated in a functional P8-C, that is, a P8-C moiety that could be efficiently incorporated into the wt phage coat and support the display of C-terminal peptide fusions. For this purpose, eight non-overlapping libraries were constructed. Each library randomized six to eight continuous positions, and together, the eight libraries spanned the entire P8-C sequence. The positions were randomized with NNS degenerate codons that encode for all 20 natural amino acids. Each library was cycled separately through two rounds of selection for binding to the Erbin PDZ domain to select for efficient C-terminal display of the PDZ ligand. Individual clones from the selected pools were sequenced, the sequences were aligned, and the distribution of the 20 natural amino acids at each position was calculated to produce a database of P8-C diversity compatible with C-terminal display (Table 2).

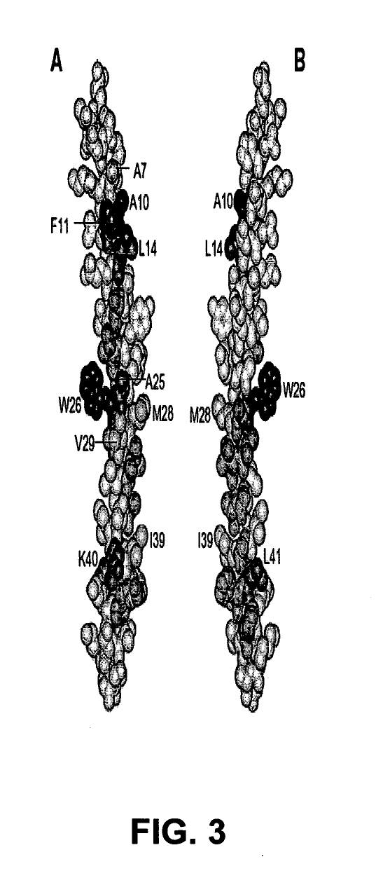

To obtain a quantitative measure of the relative diversities at each site, Shannon entropy was calculated (FIG. 2). Shannon entropy is a metric that has been used to quantify diversity in immunoglobulins and other immune system receptors (18; 19; 20). Shannon entropy ranges from a minimum of zero (completely conserved sequence) to a maximum of 4.32 (equal occurrence of all 20 amino acids). The Shannon entropy values were mapped onto the structure of P8 to obtain a structural view of the diversity distribution (FIG. 3).

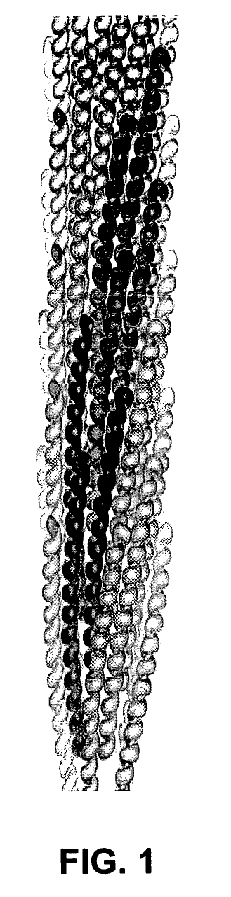

P8 is an α-helix, and the phage coat consists of interlocking layers of P8 molecules that form a sheath around the viral DNA (FIG. 1) (1). One face of the P8 helix is completely buried in the phage coat (FIG. 3A) while the other face is solvent exposed at the N-teminal end and buried at the C-terminal end (FIG. 3B). The highly conserved residues cluster into four distinct epitopes. Three of these epitopes are located along the completely buried face: a hydrophobic epitope near the N-terminus (positions 7, 10, 11 and 14), a hydrophobic epitope at the center of the helix (positions 25, 26, 28 and 29), and a positively-charged epitope near the C-terminus (positions 40, 43 and 44). A fourth hydrophobic epitope (positions 39, 41 and 42) is located directly opposite the C-terminal, positively-charged epitope. The hydrophobic epitopes pack against other P8 molecules in the phage coat, and these results indicate that these protein-protein interactions are required for efficient incorporation. Near the C-terminus, three lysine side chains (positions 40, 43 and 44) have been shown by alanine-scanning mutagenesis to be required for efficient incorporation (15), and the structure shows that these positively charged residues interact with the viral DNA core (21). The present analysis reveals that position 40 absolutely requires a positive charge, but interestingly, a positive charge was dominant but less critical at positions 43 and 44 where hydrophobic residues also occurred with significant frequency. The fourth lysine side chain at position 48 was completely dispensable, as has been demonstrated previously (15; 22). In fact, it appears that the Lys48 side chain is incompatible with C-terminal peptide display, as it did not appear among the selected sequences. On the other face of the P8 helix, there was significantly less sequence conservation (FIG. 3B). The solvent exposed N-terminal portion was highly diverse but the buried C-terminal portion was more conserved and retained an overall hydrophobic character (Table 2).

The N-terminal end (positions 1-6) was highly diverse, and this result was consistent with the fact that this region is flexible in the phage structure (1) and it has been used as a versatile fusion point for heterologous polypeptide display (7; 23). Nonetheless, this region exhibited a pronounced prevalence of negative charge and a paucity of positive charge, and this was consistent with previous reports that have demonstrated that insertion into the bacterial membrane was enhanced by negative charge and inhibited by positive charge (24). Interestingly, the C-terminal region (positions 46-60) exhibited Shannon entropy values comparable to those in the N-terminal region (FIG. 2), even amongst the last five residues of P8 (positions 46-50) which are buried in the phage particle core (1). While there was some site-specific preference for particular amino acids (e.g. aspartate or lysine at positions 56 and 58, respectively), it is notable that small glycine residues were prevalent throughout this stretch, suggesting that the C-terminal linker “squeezes” through the P8 array to become accessible for interactions on the phage surface. Thus, it appears that the last five side chains of P8 are not required for assembly into the wt phage coat, and indeed, C-terminal display can be improved by removing these side chains to form a more flexible linker sequence.

First Generation Selection for Improved C-Terminal Display

The database shown in Table 2 provides a comprehensive view of the sequence diversity compatible with C-terminal display. In addition, the best P8-C variants were also identified. Two pools of clones that had been cycled through two rounds of binding selection were formed; the N-terminal and C-terminal pools contained clones from libraries spanning positions 1-30 and 31-60, respectively. Each pool was cycled through six additional rounds of binding selection to enrich for the best C-terminal display scaffolds. DNA sequencing revealed that the N-terminal pool was dominated by clones from the libraries spanning positions 7-14 and 23-30, while the C-terminal pool was dominated by clones from the library spanning positions 45-53 (FIG. 4).

A phage ELISA was used to quantify the efficiency of C-terminal display with the P8-C variants relative to that with wt P8-C (see Materials and Methods). ELISA signal strengths depend on both phage concentration and the amount of PDZ ligand displayed on the phage particle in a form accessible for binding to the Erbin PDZ domain. The signal strengths at subsaturating phage concentrations were directly proportional to phage concentration (data not shown), and the slope of the least-squares linear fit provided a measure of the increase in ELISA signal per unit of phage concentration (15). By dividing the slope for a P8-C variant by the slope for wt P8-C, a “display enhancement” (DE) value was calculated that represented the fold increase in accessible C-terminal peptide display afforded by the selected mutations (FIG. 4). Significant display enhancements were observed for most of the selected clones, and the greatest improvement was obtained with variant P8-C-S14 which contained mutations around the fusion point between P8 and the C-terminal linker. It was speculated that the heterologous fusion produces a perturbance that inhibits incorporation into the phage coat, and display can be improved by compensatory mutations surrounding the fusion point.

It was next investigated whether mutations in different regions of P8-C could be combined to achieve further improvements in C-terminal display levels. The mutations of P8-C-S14 were combined with various mutations in the N-terminal portion of P8-C that had also improved C-terminal display. None of the combined mutants exhibited significant further improvements in display, and in fact, most exhibited reductions in display relative to P8-C-S14 (FIG. 5). These results suggest that mutations in distant regions of the P8-C molecule influence each other. Most combinations resulted in negative cooperativity, presumably because the mutations were derived from separate selections and thus were not selected to function effectively together. One might expect cooperative effects to increase with proximity, and this supposition was consistent with the observation that the mutations closer to the C-terminus (positions 23-30) were particularly ill-suited for combination with P8-C-S14 (FIG. 5).

Second Generation Selection for Improved C-Terminal Display

To further improve the levels of C-terminal display, we constructed a second generation library to select additional display-enhancing mutations in the background of the P8-C-S14 sequence. We examined the P8-C sequence diversity database (Table 2), and for each position chosen for randomization, we designed a tailored degenerate codon that ideally only encoded for abundant amino acids (FIG. 6A). We randomized a total of 16 positions in the N-terminal half of P8-C-S14 and cycled the library through four rounds of binding selection. We analyzed selected clones by DNA sequencing and phage ELISA, and the best clones exhibited greater than 100-fold display enhancements relative to wt P8-C (FIG. 6B).

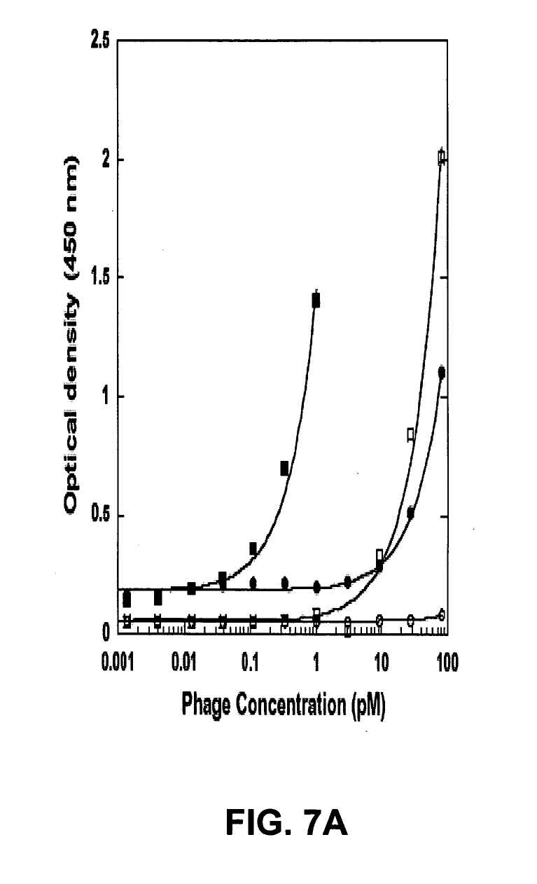

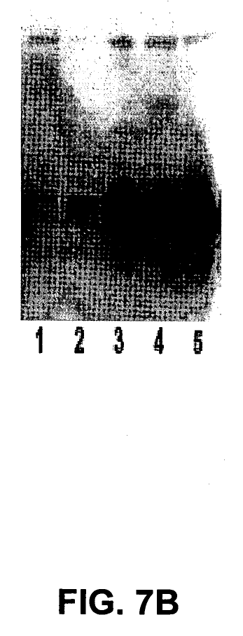

We fused a common gDtag sequence (25; 26) to the N-terminus of each P8-C moiety to investigate whether N- and C-terminal fusions could be displayed simultaneously. We used phage ELISAs to compare the levels of display achieved with wt P8-C to those achieved with the best variant selected for improved C-terminal display (P8-C-S16). Phage were captured with either the Erbin PDZ domain which binds to the C-terminally displayed peptide with moderate affinity (Kd˜100 nM) (17) or an antibody that binds to the N-terminally displayed peptide with high affinity (Kd˜1 nM) (25; 26). When displayed on the improved P8-C-S16 variant, both peptides could be detected in the low picomolar range, and the N-terminal peptide was detected more efficiently than the C-terminal peptide, as would be expected based on the relative affinities for the capturing ligands (FIG. 7A). When displayed on the wt P8-C, in contrast, the C-terminal peptide was not detected at phage concentrations approaching 100 pM, and even the high affinity N11 terminal peptide was only detected with efficiencies comparable to those for the moderate affinity C-terminal peptide displayed on the improved variant. These results are consistent with an approximately 100-fold improvement in the levels of display achieved with the P8-C-S16 variant relative to wt P8-C.

We also used western blotting of denatured phage particles to examine the relative levels of incorporation for selected P8-C variants. To standardize detection, we probed with the antibody specific for the common gDtag sequence fused to the N-terminus of each P8 moiety. As shown in FIG. 7B, the first generation mutations of P8-C-S14 substantially increased levels of display in comparison with wt P8-C (lane 2 versus lane 1). The increases in levels of display were even more dramatic for the second generation variants P8-C-S16 and P8-C-S27 (lanes 3 and 4) which exhibited bands only slightly less intense than that for wt P8 with no C-terminal fusion (lane 5). While the large differences in band intensities only allow for a qualitative comparison of the lanes, the results are again consistent with substantially greater incorporation of the P8-C variants relative to wt P8-C.

Discussion

The database compiled herein provides a comprehensive assessment of P8 sequence diversities compatible with the display of a C-terminal fusion. Overall, the results are in good agreement with previous studies which examined P8 mutations in the context of N-terminal display (10). In particular, a previous study used combinatorial alanine-scanning mutagenesis to identify functional epitopes required for the incorporation of a P8 moiety displaying an N-terminal protein fusion (15). Both studies reveal the importance of an N-terminal hydrophobic epitope (positions 7, 10, 11 and 14) and the requirement for a positively charged epitope near the C-terminus (positions 40, 43 and 44). In addition, a C-terminal hydrophobic epitope identified by alanine-scanning (positions 39, 41, 42, and 45) also appears to be important by our current analysis, as hydrophobic side chains dominate at these sites (Table 2). The major difference between the requirements for efficient N- and C-terminal display appears to be that C-terminal display was dependent upon an additional hydrophobic epitope at the center of the P8 molecule (positions 25, 26, 28 and 29) (FIG. 3A). These side chains were dispensable for N-terminal display, as evidenced by a minimized P8 variant in which 27 side chains (including Trp26, Met28 and Val29) were replaced by alanines without significantly effecting incorporation efficiency (15).

The dependence of C-terminal display on these additional side chains suggests that the C-terminal fusion has altered the assembly mechanism such that the central portion of P8 participates more directly in the process. This supposition is consistent with the observation that mutations at the C-terminus of P8 appear to exhibit cooperativity with mutations in the central region (FIG. 5), and thus, it is reasonable to suppose that C-terminal fusions may engender effects that extend far from the local perturbance. Alternatively, it is also possible that the P8-C molecules interact with each other in the phage coat, rather than being randomly distributed amongst a predominantly wt P8 array. In such a scenario, it is possible that residues in the central region of a P8 molecule could compensate for perturbations caused by C-terminal fusions displayed by neighbouring P8 molecules, as the C-terminus of P8 interacts with the central regions of other P8 molecules in the phage coat (1).

It should be noted that our interpretations assume that the P8-C variants are inserted randomly along a filament composed predominantly of wt P8 molecules. This is the most likely scenario since most of the several thousand P8 molecules in the phage coat are packed in the symmetric array covering the filament length. However, the P8 molecules near each of the asymmetric ends of the particle occupy unique environments, and it is possible that the P8-C moieties are only tolerated at one of these ends. Regardless, these variants appear to be capable of achieving high levels of C-terminal display.

The data described herein show that both termini of the P8 moiety are highly tolerant to mutations, consistent with the fact that polypeptide fusions can be supported at both the N- and C-terminal ends. By combining our knowledge of coat protein structure and function with phage display selections, we have significantly improved the capacity of P8 to act as a scaffold for phage display. Indeed, the results herein demonstrate that optimized P8 variants can even support simultaneous N- and C-terminal display (FIG. 7). This is believed to be the first demonstration of bi-terminal phage display with heterologous fusions at both ends of a coat protein. As described above, bi-terminal display format may prove useful in a variety of settings, including specialized applications such as selections for catalysts which can benefit from the proximal display of both substrates and enzymes on the same phage particle (27; 28).

### PARTIAL LIST OF REFERENCES

1. Marvin, D. A. (1998). Filamentous phage structure, infection and assembly. Curr. Opin. Struct. Biol. 8, 150-158.
2. Russel, M., Linderoth, N. A. & Sali, A. (1997). Filamentous phage assembly: variation on a protein export theme. Gene 192, 23-32.
3. Wickner, W. (1988). Mechanisms of membrane assembly: general lessons from the study of M13 coat protein and Escherichia coli leader peptidase. Biochemistry 27.
4. Marciano, D. K., Russel, M. & Simon, S. M. (1999). An aqueous channel for filamentous phage export. Science 284, 1516-1519.
5. Sidhu, S. S. (2001). Engineering M13 for phage display. Biomol. Eng. 18, 57-63.
6. Smith, G. P. (1985). Filamentous fusion phage: novel expression vectors that display cloned antigens on the virion surface. Science 228, 1315-1317.
7. Sidhu, S. S. (2000). Phage display in pharmaceutical biotechnology. Curr. Opin. Biotechnol. 11, 610-616.
8. Bass, S., Greene, R. & Wells, J. A. (1990). Hormone phage: an enrichment method for variant proteins with altered binding properties. Proteins 8, 309-314.
9. Lowman, H. B., Bass, S. H., Simpson, N. & Wells, J. A. (1991). Selecting highaffinity binding proteins by monovalent phage display. Biochemistry 30, 10832-10838.
10. Sidhu, S. S., Weiss, G. A. & Wells, J. A. (2000). High Copy Display of Large Proteins on Phage for Functional Selections. J. Mol. Biol. 296, 487-495.
11. Weiss, G. A. & Sidhu, S. S. (2000). Design and Evolution of Artificial M13 Coat Proteins. J. Mol. Biol. 300, 213-219.
12. Kang, A. S., Barbas, C. F., Janda, K. D., Benkovic, S. J. & Lerner, R. A. (1991). Linkage of recognition and replication functions by assembling combinatorial antibody Fab libraries along phage surfaces. Proc. Natl. Acad. Sci. USA 88, 4363-4366.
13. Iannolo, G., Minenkova, O., Petruzzelli, R. & Cesareni, G. (1995). Modifying filamentous phage capsid—Limits in the size of the major capsid protein. J. Mol. Biol. 248, 835-844.
14. Kretzschmar, T. & Geiser, M. (1995). Evaluation of antibodies fused to minor coat protein III and major coat protein VIII in bacteriophage M13. Gene 155, 61-65.
15. Roth, T. A., Weiss, G. A., Eigenbrot, C. & Sidhu, S. S. (2002). A minimized M13 coat protein defines the minimum requirements for assembly into the bacteriophage particle. J. Mol. Biol. 322, 357-367.
16. Fuh, G., Pisabarro, M. T., Li, Y., Quan, C., Lasky, L. A. & Sidhu, S. S. (2000). Analysis of PDZ domain-ligand interactions using carboxyl-terminal phage display. J. Biol. Chem. 275, 21486-21491.
17. Skelton, N. J., Koehler, M. F. T., Zobel, K., Wong, W. L., Yeh, S., Pisabarro, M. T., Yin, J. P., Lasky, L. A. & Sidhu, S. S. (2003). Origins of PDZ domain specificity: structure determination and mutagenesis of the Erbin PDZ domain. J. Biol. Chem. 278, 7645-7654.
18. Cowell, L. G., Kepler, T. B., Janitz, M., Lauster, R. & Mitchison, N. A. (1998). The distribution of variation in regulatory gene segments, as present in MHC class II promoters. Genome Res. 8, 124-134.
19. Stewart, J. J., Lee, C. Y., Ibrahim, S., Watts, P., Shlomchik, M., Weigert, M. & Litwin, S. (1997). A Shannon entropy analysis of immonoglobulin and T cell receptor. Mol. Immnunol. 34, 1067-1082.
20. Zemlin, M., Klinger, M., Link, J., Zemlin, C., Bauer, K., Engler, J. A., Schroeder, H. W., Jr. & Kirkham, P. M. (2003). Expressed murine and human CDR-H3 intervals of equal length exhibit distinct repertoires that differ in their amino acid composition and predicted range of structures. J. Mol. Biol. 334, 733-749.
21. Marvin, D. A., Hale, R. D., Nave, C. & Helmer-Citterich, M. (1994). Molecular models and structural comparisons of native and mutant class I filamentous bacteriophages Ff (fd, f1, M13), If1 and IKe. J. Mol. Biol. 235, 260-86.
22. Hunter, G. J., Rowitch, D. H. & Perham, R. N. (1987). Interactions between DNA and coat protein in the structure and assembly of filamentous bacteriophage. Nature 327, 252-254.
23. Smith, G. P. & Petrenko, V. A. (1997). Phage display. Chem. Rev. 97, 391-410.
24. Peters, E. A., Schatz, P. J., Johnson, S. S. & Dower, W. J. (1994). Membrane insertion defects caused by positive charges in the early mature region of protein III of filamentous phage fd can be corrected by prlA suppressors. J. Bact. 176, 4296-4305.
25. Vajdos, F. F., Adams, C. W., Breece, T. N., Presta, L. G., de Vos, A. M. & Sidhu, S. S. (2002). Comprehensive functional maps of the antigen-binding site of an anti-ErbB2 antibody obtained with shotgun scanning mutagenesis. J. Mol. Biol. 320, 415-428.
26. Lasky, L. A. & Dowbenko, D. J. (1984). DNA sequence analysis of the typecommon glycoprotein-D genes of herpes simplex virus types 1 and 2. DNA 3, 23-29.
27. Demartis, S., Huber, A., Viti, F., Lozzi, L., Giovannoni, L., Neri, P., Winter, G. & Neri, D. (1999). A strategy for the isolation of catalytic activities from repertoires of enzymes displayed on phage. J. Mol. Biol. 286, 617-633.
28. Pedersen, H., Holder, S., Sutherlin, D. P., Schwitter, U., King, D. S. & Schultz, P. G. (1998). A method for directed evolution and functional cloning of enzymes. Proc. Natl. Acad. Sci. USA 95, 10523-10528.
29. Sidhu, S. S. & Weiss, G. A. (2004). Oligonucleotide-directed construction of phage display libraries. In Phage Display: A Practical Approach (Lowman, H. B. & Clackson, T., eds.), pp. 27-41. Oxford University Press, Oxford, U.K.
30. Sidhu, S. S., Lowman, H. B., Cunningham, B. C. & Wells, J. A. (2000). Phage display for selection of novel binding peptides. Methods Enzymol. 328, 333-363.
31. Kunkel, T. A., Roberts, J. D. & Zakour, R. A. (1987). Rapid and efficient sitespecific mutagenesis without phenotypic selection. Methods Enzymol. 154, 367-382.
32. Laura, R. P., Witt, A. S., Held, H. A., Gerstner, R., Deshayes, K., Koehler, M. F., Kosik, K. S., Sidhu, S. S. & Lasky, L. A. (2002). The Erbin PDZ domain binds with high affinity and specificity to the carboxyl termini of delta-catenin and ARVCF. J. Biol. Chem. 277, 12906-12914.
33. Weiss, G. A., Watanabe, C. K., Zhong, A., Goddard, A. & Sidhu, S. S. (2000). Rapid mapping of protein functional epitopes by combinatorial alanine scanning. Proc. Natl. Acad. Sci. USA 97, 8950-8954.
34. Pearce, K. H., Jr. (1997). Mutational analysis of thrombopoietin for identification of receptor and neutralizing antibody sites. J. Biol. Chem. 272, 20595-20602.

What is claimed:

1. A fusion protein comprising a heterologous polypeptide fused to a major coat protein of a virus, wherein the major coat protein is a variant of a wild type major coat protein of the virus and is capable of C-terminal display of the heterologous polypeptide at a display level more than 30 times that of a corresponding coat protein comprising a wild type sequence.

2. A fusion protein comprising a major coat protein of a virus fused on its N-terminus to a first heterologous polypeptide and on its C-terminus to a second heterologous polypeptide, wherein the major coat protein is a variant of a wild type major coat protein of the virus.

3. The fusion protein of claim 2 wherein the variant coat protein is capable of C-terminal display of the heterologous polypeptide at a display level more than 30 times that of a corresponding coat protein comprising a wild type sequence.

4. A replicable expression vector comprising a gene fusion, wherein the gene fusion encodes the fusion protein of claim 1, 2 or 3.

5. A library comprising a plurality of the replicable expression vectors of claim 4, the expression vectors comprising a plurality of different gene fusions encoding a plurality of fusion proteins.

6. A host cell comprising the vector of claim 4.

7. A virus displaying the fusion protein of claim 1 or 2 on the surface thereof.

8. A library of virus, comprising a plurality of the virus of claim 7 displaying a plurality of different fusion proteins on the surface thereof.

9. A method, comprising:
constructing a library of phage or phagemid particles displaying a plurality of the fusion protein of claim 1 or 2;
contacting the phage or phagemid particles with a target molecule or substance; and
separating particles having a desired selection characteristic from those that do not.


SEQUENCE LISTING (21 sequences)

SEQ ID NO: 1 - protein, 50 aa, Artificial Sequence
  M13 Major Coat Protein
AEGDDPAKAAFNSLQASATEYIGYAWAMVVVIVGATIGIKLFKKFTSKAS

SEQ ID NO: 2 - protein, 50 aa, Artificial Sequence
  F1 and Fd Major Coat Protein
AEGDDPAKAAFDSLQASATEYIGYAWAMVVVIVGATIGIKLFKKFTSKAS

SEQ ID NO: 3 - protein, 50 aa, Artificial Sequence
  Zj-2 Major Coat Protein
AEGDDPAKAAFDSLQASATEYIGYAWAMVVVIVGATIGIKLFKKFASKAS

SEQ ID NO: 4 - protein, 50 aa, Artificial Sequence
  Ifl Major Coat Protein
DDATSQAKAAFDSLTAQATEMSGYAWALVVLVVGATVGIKLFKKFVSRAS

SEQ ID NO: 5 - protein, 50 aa, Artificial Sequence
  I2-2 Major Coat Protein
STATSYATEAMNSLKTQATDLIDQTWPVVTSVAVAGLAIRLFKKFSSKAV

SEQ ID NO: 6 - protein, 50 aa, Artificial Sequence
  Ike Major Coat Protein
NAATNYATEAMDSLKTQAIDLISQTWPVVTTVVVAGLVIRLFKKFSSKAV

SEQ ID NO: 7 - protein, 10 aa, Artificial Sequence
  Synthetic ERBIN binder
RAAERWDTWV

SEQ ID NO: 8 - protein, 60 aa, Artificial Sequence
  Wild-type M13 plus synthetic C-terminal linker
AEGDDPAKAAFNSLQASATEYIGYAWAMVVVIVGATIGIKLFKKFTSKASAWEENIDSAP

SEQ ID NO: 9 - protein, 50 aa, Artificial Sequence
  Synthetic S16
AEGDDPGVLAFHELQASATEYIGYAWAMVVVIVGATIGIKLFKKFTSKAS

SEQ ID NO: 10 - protein, 50 aa, Artificial Sequence
  Synthetic S17
AEGDDAAVQAFQELLHMTGQFIPMAWAMVVVIVGATIGIKLFKKFTSKAS

SEQ ID NO: 11 - protein, 50 aa, Artificial Sequence
  Synthetic S18
AEGDDYGPAAFRTLFDLAEDMVGLAWAMVVVIVGATIGIKLFKKFTSKAS

SEQ ID NO: 12 - protein, 50 aa, Artificial Sequence
  Synthetic S19
AEGDDPGVVAFGALQASATEYIGYAWAMVVVIVGATIGIKLFKKFTSKAS

SEQ ID NO: 13 - protein, 50 aa, Artificial Sequence
  Synthetic S20
AEGDDDGVAAFSMLIDLAVDFISRAWAMVVVIVGATIGIKLFKKFTSKAS

SEQ ID NO: 14 - protein, 50 aa, Artificial Sequence
  Synthetic S21
AEGDDAGTVAFQHLFAMVEDLIHRAWAMVVVIVGATIGIKLFKKFTSKAS

SEQ ID NO: 15 - protein, 50 aa, Artificial Sequence
  Synthetic S22
AEGDDAGLPAFESLFDMVEQIIYMAWAMVVVIVGATIGIKLFKKFTSKAS

SEQ ID NO: 16 - protein, 50 aa, Artificial Sequence
  Synthetic S23
AEGDDAGLQAFRTLFDLAAHVIDMAWAMVVVIVGATIGIKLFKKFTSKAS

SEQ ID NO: 17 - protein, 50 aa, Artificial Sequence
  Synthetic S24
AEGDDSGPLAFRLLIQHVEQVVAWAWAMVVVIVGATIGIKLFKKFTSKAS

SEQ ID NO: 18 - protein, 50 aa, Artificial Sequence
  Synthetic S25
AEGDDAGALAFSHLFENAVQMVGMAWAMVVVIVGATIGIKLFKKFTSKAS

SEQ ID NO: 19 - protein, 50 aa, Artificial Sequence
  Synthetic S26
AEGDDHGLLAFDNLVTLAEDLVFRAWAMVVVIVGATIGIKLFKKFTSKAS

SEQ ID NO: 20 - protein, 50 aa, Artificial Sequence
  Synthetic S27
AEGDDPAVAAFNSLQASATEYIGYAWAMVVVIVGATIGIKLFKKFTSKAS

SEQ ID NO: 21 - protein, 10 aa, Artificial Sequence
  Linker
AWEENIDSAP